SEQUENCE LISTING (44 sequences)

SEQ ID NO: 1 - DNA, 1554 bp, Amaranthus palmeri
  source 1..1554: mol_type = other DNA
atggaagagacaagcatattatcaattggaatatcattggtttgtgttgtattagtaagtgtctcatggggaattctaaattgggtatggtttaagccaaaaaggttagaaaaatgccttagaaatcaagggtttaatggaaattcttataggttattgtatggagataccaatgaaagagctaggatcaccatggaatctagattaaacccaatgcccaacctatccaatcattttcttccacattctcttgcatttttccatcacattataactaaatatgggagaagatgctttatatggaatggacctattccattggtaaacatagaagaaccatatttaataagagaaatatttatgaaaataaatgattttcaaaaagtaaagtcaaatccaatcataaagaaggttgcaccgggcttgcttttcttagagggccatgcatgggctcaacgaaggaaactcctcactcctgctttccacatcgacaagctcaagtttatggttccaacaatatgggaaagttcaaaagaaatgattaaagaatgggaagaattggcatcaaaaacaggttcatgtgaaatagaagtttggtcatctttacaaaaattatcagcagatatgatttctagagctgcttttggaagtagttatgaagaagggaagacaatttttgatcttttaactgaacaaattaagattgtagttccagtatttaattctgtttatatccctggttggaggtttttaccaaccaaaacaaatcgaagaatatcagaaatcgacagagaaatcaggattcgactaaaaggaataattgacaaaagaaagaatacaataaatagagaagagagggcaaaggatgacttgcttagtacattaattcaagacaattttggacatgcaaataaaaagcaagacataaataagttaagtattgaagaagttgtaaatgaatgtaaattattttacttagctggacaagatacaacttcttcattattgttatggacactaattatgttgggaaaacaccaaatatggcaacaaagagcaagagaagagatcatcaatgcatttggggatgccaaacctcaatttcatgagttgaatagtcttaaaatagtaaacatgatactacaagaggttctaagattatatccaccagtattagaattaaaccgtgaagttaatcaagacataaaaataggagatgtaatacttccggcaggagttttacttaatataccaattttacttttgcaacaagatgaaaaaatatggggtaaagatgcgaaagaatttaatcccgaaagatttagtgaaggaatttcaaaagctagtaaaggaaatatgtcattctttgcgttcggttggggacctagaatatgtcttggatcgaattttgctatgattgaggccaaattaactttagtcttcattctgcaaagatttgtatttgagctttcatcatcttatcgtcatgcccctttgaatgttggaactcttagacctcaatttggtgcaccaattatatttcgtcgacgataa

SEQ ID NO: 2 - protein, 517 aa, Amaranthus palmeri
MEETSILSIGISLVCVVLVSVSWGILNWVWFKPKRLEKCLRNQGFNGNSYRLLYGDTNERARITMESRLNPMPNLSNHFLPHSLAFFHHIITKYGRRCFIWNGPIPLVNIEEPYLIREIFMKINDFQKVKSNPIIKKVAPGLLFLEGHAWAQRRKLLTPAFHIDKLKFMVPTIWESSKEMIKEWEELASKTGSCEIEVWSSLQKLSADMISRAAFGSSYEEGKTIFDLLTEQIKIVVPVFNSVYIPGWRFLPTKTNRRISEIDREIRIRLKGIIDKRKNTINREERAKDDLLSTLIQDNFGHANKKQDINKLSIEEVVNECKLFYLAGQDTTSSLLLWTLIMLGKHQIWQQRAREEIINAFGDAKPQFHELNSLKIVNMILQEVLRLYPPVLELNREVNQDIKIGDVILPAGVLLNIPILLLQQDEKIWGKDAKEFNPERFSEGISKASKGNMSFFAFGWGPRICLGSNFAMIEAKLTLVFILQRFVFELSSSYRHAPLNVGTLRPQFGAPIIFRRR

SEQ ID NO: 3 - DNA, 1554 bp, Amaranthus palmeri
  source 1..1554: mol_type = other DNA
atggaagagacaagcatattatcaattggaatatctttggtttgtgtagtattagtaagtgtctcatggggaattctaaattgggtatggtttaagccaaaaaggttagaaaaatgccttagaaatcaagggtttaatggaaattcttataggttattgtatggagataccaatgaaagagctaggatgactatggaatctagattaaacccaatgcctaacctatccaatcattttcttccacattctcttgcattttaccatcacattataactaagtatgggagaagatgctttatatggaatggacctattccattggtaaacattgaagaaccaaatttagtaagagaaatatttatgaaaataaatgattttcaaaaagcaaagtcaaacccaatcataaagaaggttgcaccgggcttgcttttcttagagggccatgcatgggctcaacgaaggaaactcctcactcctgctttccacatcgataagctcaagtttatggttccaacaatatgggaaagttcagaagaaatgattaaagaatgggaagaattggtatcaaaaacaggttcatgtgaaatagaagtttggtcatctttacaaaaattatctgcagatatgatttctcgagcagcatttggaagtagttatcaagaagggaagacaatttttgatcttttaactgaacaaattaagattgttgttccagtatttaattctgtttatatccctggttggaggtttttgccaacaaaaacaaatcgaagaatatcagaaatcgacagagaaatcaggattcgactaaaaggaataatcgacaaaagaaagaatacgataaatagagaagagagggcaaaggatgacttgcttagtacattaattcaagacaattttggacatgcaaataaaaagcaagacataaataagttaaatattgaagaaattgttaatgaatgtaaattattttacttagccggacaagatacaacttcttcattattgttatggacactaattatgttgggaaaacaccaaatatggcaacaaagagcaagagaagagatcatcaatacatttggggatgccaaacctcaatttcatgagttgaatagtcttaaaatagtaaacatgatactacaagaggttctaagattatatccaccagtattagaattaaatcgtgaagttactcaagacataaaaataggagatgtaatacttccggcaggagttttacttaatataccaattttacttttgcaacaagatgaaaaaatatggggtaaagatgcgaaagaatttaatcccgaaagatttagtgaaggaatttcaaaagctagtaaaggaaatatgtcattctttgcgttcggttggggacctagaatatgtcttggatcgaattttgctatgattgaggccaaattaactttagtcttcattctgcaaaggtttgtatttgagctttcaccatcttatcgtcatgcccctttgaatgttggaactcttagacctcaatttggtgcaccaattatatttcgtcgacgataa

SEQ ID NO: 4 - protein, 517 aa, Amaranthus palmeri
MEETSILSIGISLVCVVLVSVSWGILNWVWFKPKRLEKCLRNQGFNGNSYRLLYGDTNERARMTMESRLNPMPNLSNHFLPHSLAFYHHIITKYGRRCFIWNGPIPLVNIEEPNLVREIFMKINDFQKAKSNPIIKKVAPGLLFLEGHAWAQRRKLLTPAFHIDKLKFMVPTIWESSEEMIKEWEELVSKTGSCEIEVWSSLQKLSADMISRAAFGSSYQEGKTIFDLLTEQIKIVVPVFNSVYIPGWRFLPTKTNRRISEIDREIRIRLKGIIDKRKNTINREERAKDDLLSTLIQDNFGHANKKQDINKLNIEEIVNECKLFYLAGQDTTSSLLLWTLIMLGKHQIWQQRAREEIINTFGDAKPQFHELNSLKIVNMILQEVLRLYPPVLELNREVTQDIKIGDVILPAGVLLNIPILLLQQDEKIWGKDAKEFNPERFSEGISKASKGNMSFFAFGWGPRICLGSNFAMIEAKLTLVFILQRFVFELSPSYRHAPLNVGTLRPQFGAPIIFRRR

SEQ ID NO: 5 - DNA, 1569 bp, Amaranthus palmeri
  source 1..1569: mol_type = other DNA
atggaagagaaaagcatattatcaattggaatatctttgttttgtgtaatattatttagtgtgtcatggaaaattataaattgggtatggttaaaaccaaaaaagtttgaaaaattgcttaaaaatcaagggtttgatggcaattcttataggttattgcatggagacaccaaagataaagctaagatgaccatggaagttacttctaagcctatgcctaatctatccaatgattttcttccacatactcttgcattttttcatcacactatatctaagttcggaagaaaatgcatgatatggaatgggccggttccatttgcaagcatagctgaaccggatttggtccgggaagtttttatgaagattaaccaatttcaaaagcctaagttaaaccctaccgcaaacaaggttatatccggtttgattaccttggaggggaatgcatggacccaccgaaggaaactccttacccctgctttccacttcgataaactcaagtttatggttccagcattatgggaaagtacaaaagaaatgattgaagaatgggaagaattggtatcaaaaacaggttcatgtgaaatagaagtttggtcatctttacaaaaattatcagcagatatgatttctagagctgcttttggaagtagttatgaagaagggaagacaatttttgatcttataactgaacaaattaaaattgcaattccaatgtttaattctgtttatatccccggttcgaggtttttgccaaccaagagaaatagaaggataacacaaattgatagagaaatcaagaatctactaatgggtattattgacaaaagaaagaaggcaataaaaactcaagaaaagacaaaggatgatttgcttaatacattattagagtcaagttatgagcaagaagaaaatgggcatggaaataaaaaaaatgatattaatttaaatattgaagaaattcttggtgaatgtaaattattttacctagcaggacaagatacaactgcttctttattggtatggacattaattatgttggcaaagcatcaaaattggcaacaactagcaagacaagagattttcaatgcatttggagataaaatacctcaatttcatgacttgaaccatcttaaaatagtaaatatgatactacaagaggtgctaagattatatccaccagtaacagaaacaaatcgtgaagtaatccaagacataaaattaggaaatatgatacttccagcaggtgtcgtagtgaatattccaattctacttatgcaccatgatgaaaagatatggggtaatgatgcgaaagaatttaatccagaaagatttagtgaaggaatatcaaaggctagtaaaggaaatatgtcattatttgcgtttggttggggacctaggatatgtattggatctaactttgctatgattgaggccaaactgacattggtttgcattttacaaaggtttgtatttgagctttcaccatcttatcttcacgccccttctgctattggaagtcttagaccccaatttggtgctccaatcatatttcgtccacgaatctag

SEQ ID NO: 6 - protein, 522 aa, Amaranthus palmeri
MEEKSILSIGISLFCVILFSVSWKIINWVWLKPKKFEKLLKNQGFDGNSYRLLHGDTKDKAKMTMEVTSKPMPNLSNDFLPHTLAFFHHTISKFGRKCMIWNGPVPFASIAEPDLVREVFMKINQFQKPKLNPTANKVISGLITLEGNAWTHRRKLLTPAFHFDKLKFMVPALWESTKEMIEEWEELVSKTGSCEIEVWSSLQKLSADMISRAAFGSSYEEGKTIFDLITEQIKIAIPMFNSVYIPGSRFLPTKRNRRITQIDREIKNLLMGIIDKRKKAIKTQEKTKDDLLNTLLESSYEQEENGHGNKKNDINLNIEEILGECKLFYLAGQDTTASLLVWTLIMLAKHQNWQQLARQEIFNAFGDKIPQFHDLNHLKIVNMILQEVLRLYPPVTETNREVIQDIKLGNMILPAGVVVNIPILLMHHDEKIWGNDAKEFNPERFSEGISKASKGNMSLFAFGWGPRICIGSNFAMIEAKLTLVCILQRFVFELSPSYLHAPSAIGSLRPQFGAPIIFRPRI

SEQ ID NO: 7 - DNA, 1395 bp, Amaranthus palmeri
  source 1..1395: mol_type = other DNA
atggtgaaagaaactacatctaaacccatggatcatttttcaaatgattatatccctagggtcttcccatttcatcatcaaattatcaaaaactatggtgaaaaatcatttgtatgggatggtccaattccatcaataatgataacaaacccagaaataataagagaaatattaatgaaaatttatgattttaagaaaatagtgccaactccacttatgaagaggctcaccaacgggctagttaggttggaagggaccaaatgggccaaagaaagaaagcttcttaatcctgccttccatatggagaaattaaagcatatgataccagcatttcatgcaagttgtaaggaaatgataaaagaatgggagaaaataatatcaaaaacaggaagtggagaggtagaaatgtggtcatatttacataatatgtcagcagatgcaatttctcgagcagcatttggaagtagttttcaagaaggccaaagggtttttgaacttctaagagaacatacttctattactgtacaagctttgcaatctgtctatattcctggatccaggttccttccaaccaaaaccaatagaagggaggcaaaaataatcaaagaaatggaatcattactcaaagctatgatccaaagaaggcaaaatgctataaaaaatggagaaaatgggcataaggatgacttattggatttattattgacatcaagcatagaagataatcatgtaaaagatggaaatagcaaagaatatgttaaattgggtatgaaagaaataattgaagaatgcaaaatgttttacatagctggacaagaaaccacttcttctttgttgacatggacacttattttgttaagtaagcaccaaaattggcaacaccaagcaagagaagagattctagccacatttggggatactatccctaatgatgagggcttaaaccaattgaagaaggtaagcatgatacttcaagaggttctaaggttatatccaccggtagttaccttaggtcgaaaagtttctcatgacataaaggttggagatatggatttaccagccggtgtgcaagttaaaattccaactatttttattcaccacagtgaaaaactatggggtaaagatgcaaaagagtttaacccgaatagattttcacaaggatttttgaaagccacaggaggaaatatgtgtttctttgcattcggatggggcccacgtatatgtattggctctaattttgcattacttgaggctaaaatggcattagctttgattttgccgcactttttatttgagttgtcaccgtcctacgctcatgctcctacaatcggtcagggtactctgcgccctcaatttggtgctaagttaattttacaccgtataaaccgtatttga

SEQ ID NO: 8 - protein, 464 aa, Amaranthus palmeri
MVKETTSKPMDHFSNDYIPRVFPFHHQIIKNYGEKSFVWDGPIPSIMITNPEIIREILMKIYDFKKIVPTPLMKRLTNGLVRLEGTKWAKERKLLNPAFHMEKLKHMIPAFHASCKEMIKEWEKIISKTGSGEVEMWSYLHNMSADAISRAAFGSSFQEGQRVFELLREHTSITVQALQSVYIPGSRFLPTKTNRREAKIIKEMESLLKAMIQRRQNAIKNGENGHKDDLLDLLLTSSIEDNHVKDGNSKEYVKLGMKEIIEECKMFYIAGQETTSSLLTWTLILLSKHQNWQHQAREEILATFGDTIPNDEGLNQLKKVSMILQEVLRLYPPVVTLGRKVSHDIKVGDMDLPAGVQVKIPTIFIHHSEKLWGKDAKEFNPNRFSQGFLKATGGNMCFFAFGWGPRICIGSNFALLEAKMALALILPHFLFELSPSYAHAPTIGQGTLRPQFGAKLILHRINRI

SEQ ID NO: 9 - DNA, 1470 bp, Amaranthus palmeri
  source 1..1470: mol_type = other DNA
atggaaatattattcacttatttatccatctttgctacttttttcttcctctataaaatcatccaatccctcaaaccaaacaacaaaaaacacccaccaagcccaccatcatacccattattgggccacttacaccttctaaaacccccattccaccgtacactccaatctctagcccaacgttacggcccaatcttcactctcaaattgggccttcaacgggccgtggtagtttcctccgcatgggccgccgaagaatgtttcggtcaaaacgacgtcgtttttgcaaacagaccaaaattcataataggacaacatttaggatacaaccactctatccttatctggtccccatatggggactactggcggaacctccgacgtgtcacaaccattaccatgttatccatgagacggatcaacgaagcgggtccgacccggaaacttgagatccgaaacatgataatgaaccttctagaaagtgggagtgggacccaaaaggtgaatatgaatgaagtgctgagtaaactagcaagaaattttgtaatgaggatagtaaatggaaaatcatgggaaaaaatgataataaaaccccctgaaaatttgatgactatttgtgattttttaccatttttaagatgggtagattttaaagggattgaaaaagatatgaaggaaaaacaggttgaaagagatgagtttttacagagtttggttgatgaatttagagaaagtaagaaaaaaggtgaggatttagggatgaataatactttgattgaacaattgctggatttggaacaagctgaacctgatcaatactctgatgaaactatcaaaggaatcattttggtaatgttactagcgggatcagaaaccacagcacgaaccttagaatgggcattatcaaatctcattaaccacccagaaatcctcgcgaaagcaagaacagaaatcgacctccatgtaggcaaggaacgactagtagatgattctgatctaccaaaattaacgtatacacgttgcattgtatacgaaactctaagactatttccggctgcaccacttttggtacctcatttttcttcgcaagattgcaccataggagggtatcacgtaccaaaagggacaatgcttttcgtaaatgcttgggctatacacagagacccgaccctatgggacgagcctactgtgtttaagcccgagaggttcgaaaaggagaaagaagggtttaagtttatgccctttggaattggaaggagatcttgtccggggaacaatgtggctcttaggaatgtttcattgacattggctactcttattcagtgctttgattgggaagcagccgagagtggatcaattgatctaaccgaaaagcgtggtgctggtgcgatcattgtccctaaggaaaagccattggaggctatatgtcgaccacgaccctcaatggaggatttcctcgctaaaatttag

SEQ ID NO: 10 - protein, 489 aa, Amaranthus palmeri
MEILFTYLSIFATFFFLYKIIQSLKPNNKKHPPSPPSYPLLGHLHLLKPPFHRTLQSLAQRYGPIFTLKLGLQRAVVVSSAWAAEECFGQNDVVFANRPKFIIGQHLGYNHSILIWSPYGDYWRNLRRVTTITMLSMRRINEAGPTRKLEIRNMIMNLLESGSGTQKVNMNEVLSKLARNFVMRIVNGKSWEKMIIKPPENLMTICDFLPFLRWVDFKGIEKDMKEKQVERDEFLQSLVDEFRESKKKGEDLGMNNTLIEQLLDLEQAEPDQYSDETIKGIILVMLLAGSETTARTLEWALSNLINHPEILAKARTEIDLHVGKERLVDDSDLPKLTYTRCIVYETLRLFPAAPLLVPHFSSQDCTIGGYHVPKGTMLFVNAWAIHRDPTLWDEPTVFKPERFEKEKEGFKFMPFGIGRRSCPGNNVALRNVSLTLATLIQCFDWEAAESGSIDLTEKRGAGAIIVPKEKPLEAICRPRPSMEDFLAKI

SEQ ID NO: 11 - DNA, 1699 bp, Amaranthus palmeri
  source 1..1699: mol_type = genomic DNA
attttcatcctttttttctccatcatacaaaattaatgagacggtacttcctccgtctgaaatactcgctacatttttattattgacactattcattatttaagtttattttatatgtgattaatgtgtaagaaaaaatatagtcaagtgaattcttgtttgaatcgtctaatcacctacttatatcctattaactttttataatatttagacatgtataatttaatctataaatgattgaaataatgcattggattgcgtaaaaataaaatgtagcaagtatactggaacgaaggaagtatattggtattcatatcattttaaatgtcttctacaatttattattcgatcattgtaatatctcattatatgttatttagtaacttttttttgagataaaatatgttatttagtaacttagcgtgaaattaaggcaattataaatttataatcatgtaatagtataaatcgtcatgatttaaagcctaatttacattgaatctatattaattaatttataagagatcataattcaactaaatgttaaattttaatcaaagaaatccttctacaaaatatttaataaaagggctaaaaatcataatgaatatttggtgtaataaagaaaaaaatctaccgttatgttgcatataaagtaatgcttatgcactatctaatatcttattttcaaatgataatgcgtgagaattaattttgtagagaaaatgagacgattgaagtaaaacaaataagagcattaaacatttcaagagttttttccacccaaattatactaaaattttcaatatcattcatatcattaaatatccaataccaaacgacagctaatgatttaatgaggctaaaacacttaagaaaaataccatgttacgataaagtaacatttttaagtagtttttaaataacattttgagaattttctctatatttttctcaataattctcaataagttacattttaacaagtagcatttaccttaagcgctacttttaccaaataataatattttacacaattaaaaagtgatctacatatctaataacccataattttacaacaactatcgacaactaatagctatttagacagttaacaactactcatacaactaccttgcgccgaatccgttcaaaatatactttatagttctatttatttagtatattctttttgttgtgtatctttagttaatcgattactatgcattgttaacgacttaacgtgggtgtgtcttgttggctttcagataattactgctaatacctctctctttgaagaacatatggagccggggattctctgatcgtattctcctacatgggtatgagctgccgtctatttccctccccaaactctgatcatagtttatctatgatcgggtatgatgatgatgagttctatttattgtaaggtttgttctctatccacataatattcttcaattatcaacatcattttcttgactcttattcactcataaaattctatatgttgttcttttccatctccgtttttaagcggaatattggattgatgatgatgttttttagttagcctatatatactctcatttccttcttttgtttcacttccatccctatcccttcatttacaacaaaagctctatatatacattaggaattaataatccatttctcaataaaataaaatatataa

SEQ ID NO: 12 - DNA, 1936 bp, Amaranthus palmeri
  source 1..1936: mol_type = genomic DNA
attttcatcctttttttctccatcatacaaaataaatgagactgagaattcttccaatctgaaatacttgctacatttctattattcatactattcatcgtttaatcttattttatatgtggctaatgtgtaagaaaaaatatagtcaagtggattcttgtttaaaaagtctaatcgcctactttaatcatatttactttttataatttttagatgtgtataattcaatatataaatgattgaaataacgcattgaattgcgtaaaaagtaaaatgtagtaagtatagtaaaacggaggaagtatattggtattagtatcattttacatgtcttctacaatttattattcgatcattgtaatatctcattaaatattatttagtaacttagcgtaaaattaagacaattataataatagtataaatcatcatgatttaaagcctaatttacattgaatatatattaattaatttataagagatcataattcatctagatgttacattttaatcgaagaaatccttctaaagaatatttaataaaagggctaaaaatcataatgaatatttggtgtagtaaagaaaaaatctacccttatgttgcatataaagtaatgcttatcaactatctaatgtctcatttttaaatgataatgcgtgaaaatgaattttgtagagaaaataagacgattgaagtaaaataaataagagcattaaacttgtcaagagttttttccacccaaattataagaaattttcaatatctttcatatcattaaatgtcgaatgccaaacgacagttaatgatttaatgaggctaaaacacttaagaaaaatatcatgtttcgataaagtaacattttaagtagtatccaaataagattttgagaattttctctatattttttctcacagtaagtctcatgggagacggtctattctatagacggaatccataaagcccagcccaaaatataacatagtatatttggggttataataaattatttggggctataataacttatatttggggttatagaatagtatattcgaggttataataccttacatttaagattataacataatataattgatattataataaaaaaatcagttaagggaaatgggctggcccaattttgtggtctatcctatagaccgtctcttgtgagattttgtgtttttctcaataagattacattctaacaagtaacatttaccttgagcgccacttttaccaaataataatattttacacaattaaaaagtgatctacatatctaataaccataattttacaacaactatcgacaactaatagctatttagacagttaacaactactcatacaactaccttgcgccgaatccgttcaaaatatacttttatagttctatttattagtaatattctttttgttgtgtatctttagttaatcgattactatgcattgttaacgtgggtgtgtcttgcaagctttcaaataattgcaggtacctctcactttgaagaacatacggagccgggggactctctgatcgtactctcctacatggctatgagttctatcttccctccccaaactctgatcatagtttatctatgagcgggatacactggatatgatgatgatgagttctatttattgtaaggtttgttctctatccacataatattcttcaattatcaacatcattttcttgactcttattcactcataaaattctatatgttgttcttttccatccccgtttttaagcggaatattgggttgatgatgatgttttttagttatcctatatatactctcatttccttcttttgtttcacttccacacatagtgtatatccctatcccttcatttacaacaaaagctctatatataattaggaattaataatccatttctcaataaaacaaaatatataa

SEQ ID NO: 13 - DNA, 986 bp, Amaranthus palmeri
  source 1..986: mol_type = genomic DNA
attttgtagagaaaataagacgattaaagtaaaacaaataagagcattaaacttgtcaagagttttttccacccaaattataagaaattttcaatatctttcatatcattaaatgtcgaataccaaacgacagttaatgatttaatgaggctaaaacacttaagaaaaatatcatatttcgataaagtaacattttaagtagtatccaaataagattttgagaattttctctatattttttctcaataagatacattctaacaagtactagtaacatttaccttaagcgctccttttaccaaataataatattttacacaattaaaaagtgatctacatatctaataaccataattttacaacaactatcgacaactaatagctatttagacagttaacaactactcatacaattaccttgcgccgaatccgttcaaagtatacttttatagttctatttattagtaatattctttttgttgtgtatctttagttagtcgattactatgcattgttaacgtgggtgtatcttgctggctttcagataattgcagatacctctcactttgaagaacatacagagccgggggactctctgatcgtactctcctacatggctatgagttgccgtctatcttccctccccaaactctgaccatggtttatctatgagcgggatacactatgatgagttctatttattgtaaggtttgttctctatccacataatattcttcaattatcgacaccattttcttgactcttattcactcataaaattctatatgttgttcttttccatccccgtttttaagcggaatattgggttgatgatgatgttttttagttagcctatatatactctcatttccttcttttgtttcacttccacacatagtgtatatccctatcccttcatttacaacaaaagctctatatataattaggaattaataatccatttctcaataaaacaaactatatta

SEQ ID NO: 14 - DNA, 1755 bp, Amaranthus palmeri
  source 1..1755: mol_type = genomic DNA
tttcttgtcttaacccnaatcatctccacttattactaatcaccacatactattcaattttcatcctttttttctccatcatacaaaattaatgagacggtacttcctccgtctgaaatactcgctacatttttattattgacactattcattatttaagtttattttatatgtgattaatgtgtaagaaaaaatatagtcaagtgaattcttgtttgaatcgtctaatcacctacttatatcctattaactttttataatatttagacatgtataatttaatctataaatgattgaaataatgcattggattgcgtaaaaataaaatgtagcaagtatactggaacgaaggaagtatattggtattcatatcattttaaatgtcttctacaatttattattcgatcattgtaatatctcattatatgttatttagtaacttttttttgagataaaatatgttatttagtaacttagcgtgaaattaaggcaattataaatttataatcatgtaatagtataaatcgtcatgatttaaagcctaatttacattgaatctatattaattaatttataagagatcataattcaactaaatgttaaattttaatcaaagaaatccttctacaaaatatttaataaaagggctaaaaatcataatgaatatttggtgtaataaagaaaaaaatctaccgttatgttgcatataaagtaatgcttatgcactatctaatatcttattttcaaatgataatgcgtgagaattaattttgtagagaaaatgagacgattgaagtaaaacaaataagagcattaaacatttcaagagttttttccacccaaattatactaaaattttcaatatcattcatatcattaaatatccaataccaaacgacagctaatgatttaatgaggctaaaacacttaagaaaaataccatgttacgataaagtaacatttttaagtagtttttaaataacattttgagaattttctctatatttttctcaataattctcaataagttacattttaacaagtagcatttaccttaagcgctacttttaccaaataataatattttacacaattaaaaagtgatctacatatctaataacccataattttacaacaactatcgacaactaatagctatttagacagttaacaactactcatacaactaccttgcgccgaatccgttcaaaatatacttttatagttctatttattagtatattctttttgttgtgtatctttagttaatcgattactatgcattgttaacgacttaacgtgggtgtgtcttgttggctttcagataattactgctaatacctctctctttgaagaacatatggagccggggattctctgatcgtattctcctacatgggtatgagctgccgtctatttccctccccaaactctgatcatagtttatctatgatcgggtatgatgatgatgagttctatttattgtaaggtttgttctctatccacataatattcttcaattatcaacatcattttcttgactcttattcactcataaaattctatatgttgttcttttccatctccgtttttaagcggaatattggattgatgatgatgttttttagttagcctatatatactctcatttccttcttttgtttcacttccatccctatcccttcatttacaacaaaagctctatatatacattaggaattaataatccatttctcaataaaataaaatatataa

SEQ ID NO: 15 - DNA, 1699 bp, Amaranthus palmeri
  source 1..1699: mol_type = genomic DNA
attttcatcctttttttctccatcatacaaaattaatgagacggtacttcctccgtctgaaatactcgctacatttttattattgacactattcattatttaagtttattttatatgtgattaatgtgtaagaaaaaatatagtcaagtgaattcttgtttgaatcgtctaatcacctacttatatcctattaactttttataatatttagacatgtataatttaatctataaatgattgaaataatgcattggattgcgtaaaaataaaatgtagcaagtatactggaacgaaggaagtatattggtattcatatcattttaaatgtcttctacaatttattattcgatcattgtaatatctcattatatgttatttagtaacttttttttgagataaaatatgttatttagtaacttagcgtgaaattaaggcaattataaatttataatcatgtaatagtataaatcgtcatgatttaaagcctaatttacattgaatctatattaattaatttataagagatcataattcaactaaatgttaaattttaatcaaagaaatccttctacaaaatatttaataaaagggctaaaaatcataatgaatatttggtgtaataaagaaaaaaatctaccgttatgttgcatataaagtaatgcttatgcactatctaatatcttattttcaaatgataatgcgtgagaattaattttgtagagaaaatgagacgattgaagtaaaacaaataagagcattaaacatttcaagagttttttccacccaaattatactaaaattttcaatatcattcatatcattaaatatccaataccaaacgacagctaatgatttaatgaggctaaaacacttaagaaaaataccatgttacgataaagtaacatttttaagtagtttttaaataacattttgagaattttctctatatttttctcaataattctcaataagttacattttaacaagtagcatttaccttaagcgctacttttaccaaataataatattttacacaattaaaaagtgatctacatatctaataacccataattttacaacaactatcgacaactaatagctatttagacagttaacaactactcatacaactaccttgcgccgaatccgttcaaaatatacttttatagttctatttattagtatattctttttgttgtgtatctttagttaatcgattactatgcattgttaacgacttaacgtgggtgtgtcttgttggctttcagataattactgctaatacctctctctttgaagaacatatggagccggggattctctgatcgtattctcctacatgggtatgagctgccgtctatttccctccccaaactctgatcatagtttatctatgatcgggtatgatgatgatgagttctatttattgtaaggtttgttctctatccacataatattcttcaattatcaacatcattttcttgactcttattcactcataaaattctatatgttgttcttttccatctccgtttttaagcggaatattggattgatgatgatgttttttagttagcctatatatactctcatttccttcttttgtttcacttccatccctatcccttcatttacaacaaaagctctatatatacattaggaattaataatccatttctcaataaaataaaatatataa

SEQ ID NO: 16 - DNA, 1383 bp, Amaranthus palmeri
  source 1..1383: mol_type = genomic DNA
tagggtctattatcatttcttgtcttaaacccccaaatcatctccacttattactaatcaccacatactattcaattttcatcctttttttctccatcatacaaaatgaatgagtatgaattttgtagagaaaatgagacgattaaagtaaaacaaaaaagaatattaaacttgtttagagttttttccactcaaattatactaaatttttaatatcattaaatatcgaataccaaacgccggttaatgatttgatgaggcaaaaacacttaagaaaaataccatgtttcaataaagtaacattttaagtagtattcaaatacacaaattcttacttgagaccgtcttttgtaaagacgactctcaaaagtccagtccaatgctaaatatatattgttacaactctaaatataaatttgggctggacttttaagagtcttctttataaaagatggtctctcaagagatccaaataacattttgagaattttctctatatttttctacataagttacattctaacaagcaacattaaccttaagcgccacttttacctaataataatattttacacaactaaaaagagatctacatatctaataactataattttacaacatttatcgacaactaatagctatatagacggttaacaattactcataaaactatgttacgcggaatcagttcagaatatacttttatagttctattcactacaatataatttgcttttagtgggatatatttagcggcatttaatgtaaatgtcgcgatttcaaatttcttataatatatatagaggatttagtaacatttattagaccctttaacagcataataaaagtgacactaaaggtttttgcgacataggatttagtgacatttctcacaaatgccactctaaactatagtgataattacatatgccactaaagaccaaataaagtgtcgctaaatcacttaatagtgaaacttaaataaaaaccaatgtttcttacattaaaaaatactccttattgacatttttttaatgccactaaatccaatgtcgcaaaaagttaaatttgtggtattgatttatagtataaggtttgttctctatccacataatattcttcaattatcaacatcattttcttgactcttattcactcataaaattctatatgttattctttccaatccctgttttcgagcgggatattggaataatgatgatcacgttgttgttttcaatactcttcaatgttttttagttagcctatatatactctcatttccttcttttgtttcacttccatccctatcccttcatttacaacaaaagctctatacattaggaattaataatccatttctcaataaaacaaaatatataa

SEQ ID NO: 17 - DNA, 1389 bp, Amaranthus palmeri
  source 1..1389: mol_type = genomic DNA
ttgatttagggtctcttatcatttcttgtcttaaaccccaaatcatctccacttattactaatcaccacatactattcaattttcatcctttttttctccatcatacaaaatgaatgagtatgaattttgtagagaaaatgagacgattaaagtaaaacaaaaaagaatattaaacttgtttagagttttttccactcaaattatactaaatttttaatatcattaaatatcgaataccaaacgccggttaatgatttgatgaggcaaaaacacttaagaaaaataccatgtttcaataaagtaacattttaagtagtattcaaatacacaaattcttacttgagaccgtcttttgtaaagacgactctcaaaagtccagtccaatgctaaatatatattgttacaactctaaatataaatttgggctggacttttaagagtcttctttataaaagatggtctctcaagagatccaaataacattttgagaattttctctatatttttctacataagttacattctaacaagcaacattaaccttaagcgccacttttacctaataataatattttacacaactaaaaagagatctacatatctaataactataattttacaacatttatcgacaactaatagctatatagacggttaacaattactcataaaactatgttacgcggaatcagttcagaatatacttttatagttctattcactacaatataatttgcttttagtgggatatatttagcggcatttaatgtaaatgtcgcgatttcaaatttcttataatatatatagaggatttagtaacatttattagaccctttaacagcataataaaagtgacactaaaggtttttgcgacataggatttagtgacatttctcacaaatgccactctagactatagtgataattacatatgccactaaagaccaaataaagtgtcgctaaatcacttaatagtgaaacttaaataaaaaccaatgtttcttacattaaaaaatactccttattgacatttttttaatgccactaaatccaatgtcgcaaaaagttaaatttgtggtattgatttatagtataaggtttgttctctatccacataatattcttcaattatcaacatcattttcttgactcttattcactcataaaattctatatgttattctttccaatccctgttttcgagcgggatattggaataatgatgatcacgttgttgttttcaatactcttcaatgttttttagttagcctatatatactctcatttccttcttttgtttcacttccatccctatcccttcatttacaacaaaagctctatacattaggaattaataatccatttctcaataaaacaaaatatataaa

SEQ ID NO: 18 - DNA, 1383 bp, Amaranthus palmeri
  source 1..1383: mol_type = genomic DNA
tagggtctcttatcctttcttgtcttaaacccccaaatcatctccacttattactaatcaccacatactattcaattttcatcctttttttctccatcatacaaaatgaatgagtatgaattttgtagagaaaatgagacgattaaagtaaaacaaaaaagaatattaaacttgtttagagttttttccactcaaattatactaaatttttaatatcattaaatatcgaataccaaacgccggttaatgatttgatgaggcaaaaacacttaagaaaaataccatgtttcaataaagtaacattttaagtagtattcaaatacacaaattcttacttgagaccgtcttttgtaaagacgactctcaaaagtccagtccaatgctaaatatatattgttacaactctaaatataaatttgggctggacttttaagagtcttctttataaaagatggtctctcaagagatccaaataacattttgagaattttctctatatttttctacataagttacattctaacaagcaacattaaccttaagcgccacttttacctaataataatattttacacaactaaaaagagatctacatatctaataactataattttacaacatttatcgacaactaatagctatatagacggttaacaattactcataaaactatgttacgcggaatcagttcagaatatacttttatagttctattcactacaatataatttgcttttagtgggatatatttagcggcatttaatgtaaatgtcgcgatttcaaatttcttataatatatatagaggatttagtaacatttattagaccctttaacagcataataaaagtgacactaaaggtttttgcgacataggatttagtgacatttctcacaaatgccactctagactatagtgataattacatatgccactaaagaccaaataaagtgtcgctaaatcacttaatagtgaaacttaaataaaaaccaatgtttcttacattaaaaaatactccttattgacatttttttaatgccactaaatccaatgtcgcaaaaagttaaatttgtggtattgatttatagtataaggtttgttctctatccacataatattcttcaattatcaacatcattttcttgactcttattcactcataaaattctatatgttattctttccaatccctgttttcgagcgggatattggaataatgatgatcacgttgttgttttcaatactcttcaatgttttttagttagcctatatatactctcatttccttcttttgtttcacttccatccctatcccttcatttacaacaaaagctctatacattaggaattaataatccatttctcaataaaacaaaatatataa

SEQ ID NO: 19 - DNA, 1407 bp, Amaranthus palmeri
  source 1..1407: mol_type = genomic DNA
tgatttagggtctcttatcatttcttgtcttaaacccccaaatcatctccacttattactaatcaccacatactattcaattataatcctttttttctccatcatacaaaatgaatgagtatgaattttgtagagaaaatgagacgattaaagtaaaataaataagaatattaagtttgtcaagagttttttccactcaaattatactaaatttttaatatcattaaatatcgaataccaaacgccaaacgccaaacgccagttaatgatttgatgaggcaaaaacacttaagaaaaataccatgtttcaataaagtaacattttaagtagtatttaaatacacaaattcttacttgagactgtcttttataaagacgattctcaaaagtccagtccaattctaaatatatattgttacaactctaaatataaatttggactggacttttgagagtcttctttataaaagagggtctctcaagagatccaaataacattttgagaattttctctatatttttctacataagttacattctaacaagcaacattaaccttaagcgccacttttacctaataataatattttacacaactaaaaagagatctacatatctaataactataattttacaacatttatcgacaactaatagctatatagacggttaacaattactcataaaactatgttacgcggaatcagttcagaatatacttttatagttctattcactacaatataatttgcttttagtgggatatatttagcggcatttaatgtaaatgtcgcgatttcaaatttcttataatatatatagaggatttagtaacatttattagaccctttaacagcataataaaagtgacactaaaggtttttgcgacataggatctagtgacatttttcacaaatatcactatagactatagtgataattacatatgccataaagaccaaataaagtgtcgctaaatcacttaatagtgaaacttaaataaaaaccaatgtttattacattaaaaatactccttattggcatttttttaatgccactaaatccaatgtcgcaaaaagttaaatttgtggtattgatttatagtatnaatttatagtataaggtttgttctctatccacataatattcttcaattatcaacatcattttcttgactcttattcactcataaaattctatatgttgttcttttccatccctgttttcgaccgaaatattgagttgatgatgatgttgttttcaatactcttcaatcttgtttagttagcctatatatactctcatttccttcttttgtttcacttccatccctatcccttcatttacaacaaaagctctatacattaggaattaataatccatttctcaataaaacaaaatataaaa

SEQ ID NO: 20 - DNA, 1341 bp, Amaranthus hypochondriacus
  source 1..1341: mol_type = other DNA
atgcctaacctatccaatgattttcttccacattctcttgcatttttccatcacattataactaagtatggaagaaaatgctttatatggaatggacctattccattggtaaacatagtagaaccatatttaataagagaaatatttatgaagataaatgattttcaaaaagcaaagtcaaacccactcataaagaaggttgcaccgggcttgattttcttagagggccatgcatgggctcaacgaaggaaactcctcactcctgctttccacatcgataagctcaagtttatgattccagcaatgtgggaaagtttaaatgagatgattaaagagtgggaagatttgacgtcaaaaagaggatcatgtgaaatagaagtgtggtcatcattacaaaaattatctgcagatatgatttctcgagcagcttttggaagtagttatcaagaagggaagaccatttttgatcttataactcaacaaattaagattgtgattcctgtatttaattctgtttatatccctggttggaggtttttgccaaccaaaacaaatcgaagaatatcagagattgatagagaaattaggaatcgattaaaagatataattgacaaaagaaagaagtcaataaatacaaaagaaagggaaaaggatgacttgcttagtacattacttcaagacaattttggacatgcaaataaaaagcaagacataaaaaagttaaatattgaagaagttgttaatgaatgtaaattattttacttagccggacaagatacaacttcttcattattgttatggacactaattatgttgggaaagcaccaaatatggcaacaaagcgcaagagaagagatcatcaatgcatttggggatggcaaacctcaatttcatgagttgaatagtcttaaaatagtaaacatgatactacaagaggttctaagattatatccaccaatattagaattaaatcgtgaagttactcaagacataaaaataggagatgtaatacttccggcaggagttttacttaatataccaattttacttttgcaacaagatgaaaaaatatggggtaaagatgcgaaagaatttaatcccgaaagatttagtgaaggaatttcaaaggctagtaaaggaaatatgtcattctttgcattcggttggggacctagaatatgtcttggatcgaattttgctatgattgaggccaaattaacattagtcttcattctacaaaggtttgtatttgagctttcaccatcttatcgtcatgcccctttaaatgttggaactcttagacctcaatttggtgctccaattatatttcgtcgacgataa

SEQ ID NO: 21 - protein, 446 aa, Amaranthus hypochondriacus
MPNLSNDFLPHSLAFFHHIITKYGRKCFIWNGPIPLVNIVEPYLIREIFMKINDFQKAKSNPLIKKVAPGLIFLEGHAWAQRRKLLTPAFHIDKLKFMIPAMWESLNEMIKEWEDLTSKRGSCEIEVWSSLQKLSADMISRAAFGSSYQEGKTIFDLITQQIKIVIPVFNSVYIPGWRFLPTKTNRRISEIDREIRNRLKDIIDKRKKSINTKEREKDDLLSTLLQDNFGHANKKQDIKKLNIEEVVNECKLFYLAGQDTTSSLLLWTLIMLGKHQIWQQSAREEIINAFGDGKPQFHELNSLKIVNMILQEVLRLYPPILELNREVTQDIKIGDVILPAGVLLNIPILLLQQDEKIWGKDAKEFNPERFSEGISKASKGNMSFFAFGWGPRICLGSNFAMIEAKLTLVFILQRFVFELSPSYRHAPLNVGTLRPQFGAPIIFRRR

SEQ ID NO: 22 - DNA, 1557 bp, Beta vulgaris
  source 1..1557: mol_type = other DNA
atggaaacaacaagcatatctattgcagtttcattgggttgtgcagtattgttgagtgtgttatggagtgtattaaactgggtgtggttgaagcctaagagattagagatataccttaggaagcaaggcttccaaggtaatccttataggctattgcatggagataccaaagacagagctagaatgatgttggaggccagaactaaacctatgccatgtctgtcaaatcattatcttgcacgtgttctccctttcttctctcaaactatagcaaaatacggtacaagatgcttcgtatggaatggaccaataccgctggtatacatttcagaaccagcgatgataagggaagtattgatgaaaataaaccaatttcagaagccaaagacgaacccaattttgaagaaggtggcctctggcttgattcttttagagggtcaagtttgggccaaccgaaggaagcttcttaacccagctttccacatggacaagttaaagtttatgattcccgctctatgggaaagctctgatgctatggtgaaagagtgggaagtaatggtatcaaagacaggctcatgtgaaatagaagtgtggtcttgtttgcacaagctatctgcagatctcatttctagagctgcatttggaagttcttatgaagaagggaagaggatatttgaactcctcacagagcaaattaagattgcagtgccagttttggactcagtttatatcccaggatggagcttgatcccaaacaaggcaaataggaggatagcggagattgacagggaaattcgcactttgctaggaggtgttattgacaagaggaagaaactaataaatgcaggagaaaagcctaaggatgacttactgggtttactattggagtcaagtttcaatgaaattcaacatggaaataacaagcgaggcatgaagttgaatatagaagatgtagtgaatgagtgtaagttattctacttaggaggacaagatacgacttctacattgctcgtatggacactaattcttttgggaaagcaccaaaattggcaagcacgagctcgagaagaggtcttgaacacgtttggaaatcgcattcctgattttcatggattgaatcatctaaaaattgttaacatgatacttcaagaggtccttagattatatcctccagctgtggaattaaatcgcgtagtaagtgaagatatgacaatcggagatgtgtttttaccagcaggtgtgttagttaatcttccaatactcctcatccaacaagatgaaaagttatggggtgaagatgctaaagaatttaatcctgaaagatttaatgaaggtatcagcaaagcaactaaagggaacatgtcatttttctcatttggttggggaccaagaatatgcattggatcaaattttgccatgactgaagctaaaatgacattagccttgattctacaacgattcgtctttgagttgtctccatcatatgctcatgctccatctggtactggaactcttcgacctcaattcggtgctcaaattatacttaagagactatag

SEQ ID NO: 23 - protein, 518 aa, Beta vulgaris
METTSISIAVSLGCAVLLSVLWSVLNWVWLKPKRLEIYLRKQGFQGNPYRLLHGDTKDRARMMLEARTKPMPCLSNHYLARVLPFFSQTIAKYGTRCFVWNGPIPLVYISEPAMIREVLMKINQFQKPKTNPILKKVASGLILLEGQVWANRRKLLNPAFHMDKLKFMIPALWESSDAMVKEWEVMVSKTGSCEIEVWSCLHKLSADLISRAAFGSSYEEGKRIFELLTEQIKIAVPVLDSVYIPGWSLIPNKANRRIAEIDREIRTLLGGVIDKRKKLINAGEKPKDDLLGLLLESSFNEIQHGNNKRGMKLNIEDVVNECKLFYLGGQDTTSTLLVWTLILLGKHQNWQARAREEVLNTFGNRIPDFHGLNHLKIVNMILQEVLRLYPPAVELNRVVSEDMTIGDVFLPAGVLVNLPILLIQQDEKLWGEDAKEFNPERFNEGISKATKGNMSFFSFGWGPRICIGSNFAMTEAKMTLALILQRFVFELSPSYAHAPSGTGTLRPQFGAQIILKRL

SEQ ID NO: 24 - DNA, 1548 bp, Spinacia oleracea
  source 1..1548: mol_type = other DNA
atggaagtaacaagaatatcaataggaatttcgttggtttttgttgtattagttagtgtattatggtctattttaaattggatatggtttaagccaaagagattagagaaatgccttaggaaccaagggtataaaggtaattcttataggcttttgcatggtgacaccaaagatagagcttctatgactatggaagctagatcacagcctcctttgccacttatgtccaatgatattcttccacgtgttgtcccgttcttcattcacactcttaacacatatggaagaagatgctttgtatggaatggtccagtgccgttggtgaccattgcagaaccagagcttataagggaagtgttaatgaagatacacgagtttcaaaagccagtaacaaatccactgttaaagaaagttgcctctggtttggttatgttagagggtcaaatgtgggctaatcggaggaagctcctcactcctgctttccacatggataagttaaagtatatgattcccgcattatgggaaagctctaattgtatggtcaaggagtgggaagaaatggtatcggagaaaggttcgcttgaaattgaagtgtggtcacatttgcatatattatcagctgatctcatttctagagctgcgtttggaagtagttatgaagaagggaagagaatatttgaactcttaactgaacaaattcagattgcggttccagttttcaattcagtttatattcctggttggagatttgtcccaaccaaggcaaaccgaaggatagtggaacttgacagggaaatccgcaccctactaaaaggtgttattgaaaagagaaagcaagcaattaaagcaggagaaaagccaaaggatgacttattaggaatactattggagtcaaattttgtaggcattggacacggaaataaaaaacaattacaacatcgcattgagttaacaattgaagaagtgataaatgaaagtcaaggcaccacttctacgttacttgtatggacactaatcatgttggcaaggcatcaaaactggcaagctcgagctcgtgaagaggttttaaacacgtttggaaatcgtaaccctgactttcaaggactaaatcatcttaaaattgttaacatgatactacaagaggtacttaggttatatcccccggttttagaattaaaccgtacagttaacaaagatattagagtcggagatgtcttcttacctgcaggtgtgttagttaacctcccaatactccttatacaacaagatgaaaaactatggggtgatgatgctaaagaatttaagccagaaaggtttagtgaaggaatctcgaaggcgacgaaagggaacatgtcatttttctcatttgggtggggcccaagaatatgcattggatcaaattttgccatgattgaagccaaaatgacattagctttgattctacaacgtttctcttttgaactttcgccgtcttatgctcatgctccatccgccattggagcacttaaacctcaattcgacgctccaatcatcttcaacaaactatag

SEQ ID NO: 25 - protein, 515 aa, Spinacia oleracea
MEVTRISIGISLVFVVLVSVLWSILNWIWFKPKRLEKCLRNQGYKGNSYRLLHGDTKDRASMTMEARSQPPLPLMSNDILPRVVPFFIHTLNTYGRRCFVWNGPVPLVTIAEPELIREVLMKIHEFQKPVTNPLLKKVASGLVMLEGQMWANRRKLLTPAFHMDKLKYMIPALWESSNCMVKEWEEMVSEKGSLEIEVWSHLHILSADLISRAAFGSSYEEGKRIFELLTEQIQIAVPVFNSVYIPGWRFVPTKANRRIVELDREIRTLLKGVIEKRKQAIKAGEKPKDDLLGILLESNFVGIGHGNKKQLQHRIELTIEEVINESQGTTSTLLVWTLIMLARHQNWQARAREEVLNTFGNRNPDFQGLNHLKIVNMILQEVLRLYPPVLELNRTVNKDIRVGDVFLPAGVLVNLPILLIQQDEKLWGDDAKEFKPERFSEGISKATKGNMSFFSFGWGPRICIGSNFAMIEAKMTLALILQRFSFELSPSYAHAPSAIGALKPQFDAPIIFNKL

SEQ ID NO: 26 - DNA, 1506 bp, Chenopodium quinoa
  source 1..1506: mol_type = other DNA
atggaaggaaaaggaataatgtttgtagtattttctttggtatgtgttgtgttgctgaatttgttgtggagtatattgaagtgggtatggttgaagcctaaaagattagaaagatgtctcagaaagcaaggtttccatggtaactcttataggctactctatggagataccaaagacaaagaaaggatgctgattgaggctagatcgaaacccgctatgccgtatttatcaaatgatcatctttcacgtgttcttccattctttcaacacactgtcaatacatacggtaaaagatgttttatgtggaatggtccaacgcccttggttacgatttcggaaccagaattgatgagagaagtgtttatgaagataaacgagtttcagaagccacaatcgaaccatttttttaagcagctcgcaactggtttggttcaagtagagggtcatgtatgggccaagcggaggaagctactcaatccagcttttcacattgacaagctaaagtttatgcttcctgcattttggacaagttcatatcacatgatccaagaatgggaagaacaggcgtcagagacggggtcttgtgagatagaagtatggtcacatttgcataagctatctgcagatgtcatttcaagagctgcgtttggaagtagttatgaagaagggaaaaaaatatttgaattcataaacgagcaaattatggttgccatgccacttataaattcagtttatatccctggattgaggtttgtcccaactagggcacataagaggatggcggacattgacaaacaaatccgcactctactgaaaggtattattgacaatcgtaagaaagcgatgaagatgggagaaaaatcgaaggatgatttgttgggtttattactaaactcaagtttccaagatattcaaaatggaagtaagaagcaaaatttgactaagttggatcttaaagaagtgattgatgaatgcaagatattctacttagcaggccaagaaactacttccacattacttgtatggacattaattttacttggaaaacatcaagaatggcaagcacgagcacgagaagaggtaaacaagattctacaggaagttcttagattatatccccctgtgtctgagataagtcgcacagttaaagaagacatcaaagttggagaaatcttcttgcctaaaggagtgttaatcaatcttccaatactacttgtacatcaagacgaaaagtattggggacctgatgctaaggagttcaagcctgagagatttagtgaaggaatttgtaaggcgagcggagggaacatgtcatttttctcatttggttgggggccaagaatatgcattgggtctaattttgccatgatggaagctaaattgacattagctttaattttgcaacgcttctcttttaacctttcaccattgtatgctcatgcaccttcagctcttggaactcttcgtcctcagtttggtgctccgataatattttatcgactatag

SEQ ID NO: 27 - protein, 501 aa, Chenopodium quinoa
MEGKGIMFVVFSLVCVVLLNLLWSILKWVWLKPKRLERCLRKQGFHGNSYRLLYGDTKDKERMLIEARSKPAMPYLSNDHLSRVLPFFQHTVNTYGKRCFMWNGPTPLVTISEPELMREVFMKINEFQKPQSNHFFKQLATGLVQVEGHVWAKRRKLLNPAFHIDKLKFMLPAFWTSSYHMIQEWEEQASETGSCEIEVWSHLHKLSADVISRAAFGSSYEEGKKIFEFINEQIMVAMPLINSVYIPGLRFVPTRAHKRMADIDKQIRTLLKGIIDNRKKAMKMGEKSKDDLLGLLLNSSFQDIQNGSKKQNLTKLDLKEVIDECKIFYLAGQETTSTLLVWTLILLGKHQEWQARAREEVNKILQEVLRLYPPVSEISRTVKEDIKVGEIFLPKGVLINLPILLVHQDEKYWGPDAKEFKPERFSEGICKASGGNMSFFSFGWGPRICIGSNFAMMEAKLTLALILQRFSFNLSPLYAHAPSALGTLRPQFGAPIIFYRL

SEQ ID NO: 28 - DNA, 1560 bp, Solanum tuberosum
  source 1..1560: mol_type = other DNA
atggattgtgttcaatttatagtaatatcatcttgttctgccatagcgattactctattgttgtgtctatggagagttctgaactggatttggttcaggccgaagaaattggagaagttactgaaagaacaaggcgtgaagggcaattcgtactcgatattgtatggggacacaaaagaactttctctgaggattaaagaagttacatccaaatccatgagtctttacgatgatgatatagcacaaagatcgatgcctttctttcttgacaccatcaagaaatatgggaagaaatcatttatatggttgggtccaaaaccactggtgttcatcatggatcctgaccttataaaggaagtattcacaaaaacatacatttatcaaaagacgaaaacaaatccgttaaccaagttacttgcacaaggactagtaagagaggagggagacaaatgggctaaacatagaaaaatcatcaatcccgccttccatctagagaagctaaagcatatgcttccagctttttgtttgagctgtactgagatgctaagtaaatgggaagacattgtttcggttgagagagcatacgagatagatgtatggcctcaccttcaacaattgactagtgatgtgatctctcgaacagcttttggcagtagttacgaagaaggtagaagaatatttgaacttcaaacagaacaagctcagtatttcattgaagctgtacgctcggtttatatcccagggtggagttttttgccaacaaagaggaatcgaagaatgaaggaaatcgaaaggcatgttcaggctacaattagaggtattattgataaaagagtgaaggcaatgaaaggaggagaggcgaataacgaggatctacttggtatattgctcgaatcaaattttaaagaaatcgaacagcatggagacaagaatttcggacttagcattgaagaagtcattgaagaatgcaagttgttctattttgctggacaagaaactacatcggcgttgcttgtatggactctaattttgttgtgcaggcatcatgattggcagacacgggcaagagaagaggtgttgcaagtgtttgggaatcggaaaccagattttgatggactgaatcatctaaaagttgtgacaatgatcttgtacgagtcgttaaggttataccctccagtaatgacagttacacgccggcttaatgaagacactgtactaggagatatgtctctaccagcaggtgtgctactctccttaccagcggtcttattacatcacgatgaagagatatggggtgaagatgcaaagaaattcaatccagagagatttagagaaggtgtctcaagtgcaacgaagggtcaagtcactttcttcccatttacatggggtcctagaatatgcattggacaaaacttcgccatgttagaagcaaagatggtgttggctatgatcctacaacgtttctcgttcgatttatcttcatcttatgctcatgctcctcattccgttataactacgcaaccccaatatggtgctccacttattctgcacagactatag

SEQ ID NO: 29 - protein, 519 aa, Solanum tuberosum
MDCVQFIVISSCSAIAITLLLCLWRVLNWIWFRPKKLEKLLKEQGVKGNSYSILYGDTKELSLRIKEVTSKSMSLYDDDIAQRSMPFFLDTIKKYGKKSFIWLGPKPLVFIMDPDLIKEVFTKTYIYQKTKTNPLTKLLAQGLVREEGDKWAKHRKIINPAFHLEKLKHMLPAFCLSCTEMLSKWEDIVSVERAYEIDVWPHLQQLTSDVISRTAFGSSYEEGRRIFELQTEQAQYFIEAVRSVYIPGWSFLPTKRNRRMKEIERHVQATIRGIIDKRVKAMKGGEANNEDLLGILLESNFKEIEQHGDKNFGLSIEEVIEECKLFYFAGQETTSALLVWTLILLCRHHDWQTRAREEVLQVFGNRKPDFDGLNHLKVVTMILYESLRLYPPVMTVTRRLNEDTVLGDMSLPAGVLLSLPAVLLHHDEEIWGEDAKKFNPERFREGVSSATKGQVTFFPFTWGPRICIGQNFAMLEAKMVLAMILQRFSFDLSSSYAHAPHSVITTQPQYGAPLILHRL

SEQ ID NO: 30 - DNA, 1560 bp, Solanum lycopersicum
  source 1..1560: mol_type = other DNA
atggattatgttcaatttatagtaaaagcgtcttgttctgttatagcgattactctgttgttgtgtatatggagcgttctgaactggatttggttcaggccgaagaaattggagaagctgctgaaagaacaaggtgtgaagggcaattcgtactcgattttgtatggggacacaaaagaactttccatgaggattaaagaagttacatccaaatccatgagtgtttacgatgatgatatagcaccaagatcgttgcctttctttcttgacaccatcaagaaatacgggaagaaatcatttatatggttgggtccaaaaccactggtgttcatcatggatcctgatctcataaaagaagtattcacaaaaacatacatttaccaaaagacgaaaacaaatccattgaccaagttacttgcacaaggactagtaagagaggagggagacaaatgggctaaacacagaaaaatcatcaatcccgccttccatctagagaaactaaagcatatgcttccagctttttatttgagctgtactgagatgctgagtaaatgggaagatgttgtttcggttgagagatcacacgagatagatgtatggcctcaccttcagcaattgactagtgatgtgatctctcgaacagcttttggcagtagctatgaagaaggtagacgaatatttgaacttcaaacagaacaagctcagtatttcattgaagctgtacgctccgtttatatcccagggtggagttttttgccaacaaagaggaatcgaagaatgaaggaaatcgagaagcatgttcaggctacaattagaggaattattgataaaagagtgaaggcaatgacaggaggagaggcgaataacaaggatctacttggtatattgctcgaatcaaattttaaagaaatcaaacaacatggagacaaggactttggacttagcatcgaagaagtcattgaagaatgcaagttgttctattttgctggacaagaaactacatcggcgttgcttgtatggactctaattttgttgtgcagacatcacgattggcagacacgggccagaacagaggtattgcaagtgtttgggagtcggaaaccagattttgatggactgactcatctaaaagtagtgacaatgatcttgtacgaatcgttaaggttataccctccagtaacgacagttacacgccggcttaatgaagatgctgtactaggagatatatctctaccagcagacgtgctactctcattaccagcggtcttattacaccacgatgaagagatatggggtgaagacgcaaagaaattcaatccagagagatttagagaaggcgtttcaagtgcaacaaagggtcaagtcactttcttcccgtttacatggggtcctagaatatgcattggacaaaacttcgctatgttagaagcaaagatggtgttggctatgatcctacaacgtttctcattggatttatccacatcttatactcatgctcttcattccgttataactacgcaaccccaatatggtgctccacttattttacacaaactatag

SEQ ID NO: 31 - protein, 519 aa, Solanum lycopersicum
MDYVQFIVKASCSVIAITLLLCIWSVLNWIWFRPKKLEKLLKEQGVKGNSYSILYGDTKELSMRIKEVTSKSMSVYDDDIAPRSLPFFLDTIKKYGKKSFIWLGPKPLVFIMDPDLIKEVFTKTYIYQKTKTNPLTKLLAQGLVREEGDKWAKHRKIINPAFHLEKLKHMLPAFYLSCTEMLSKWEDVVSVERSHEIDVWPHLQQLTSDVISRTAFGSSYEEGRRIFELQTEQAQYFIEAVRSVYIPGWSFLPTKRNRRMKEIEKHVQATIRGIIDKRVKAMTGGEANNKDLLGILLESNFKEIKQHGDKDFGLSIEEVIEECKLFYFAGQETTSALLVWTLILLCRHHDWQTRARTEVLQVFGSRKPDFDGLTHLKVVTMILYESLRLYPPVTTVTRRLNEDAVLGDISLPADVLLSLPAVLLHHDEEIWGEDAKKFNPERFREGVSSATKGQVTFFPFTWGPRICIGQNFAMLEAKMVLAMILQRFSLDLSTSYTHALHSVITTQPQYGAPLILHKL

SEQ ID NO: 32 - DNA, 1557 bp, Gossypium hirsutum
  source 1..1557: mol_type = other DNA
atggaagtttaccatgtaattagagatttggtctcattgcttgttgttggtttattaatgatatggggatggagagctttaaactgggtatggttggccccaaagaggctcgaaaggtgcttgagacaacagggttttgcagggaatccttacaggttcctttctggagacataaaagagctctcaactatgagcagacaaacaagagccaaacctatgcctcttagcgatgatattgggccttatgttgctccttttcttcatcaaactgtcaaccagtatggaaagaattcatttacgtggattggtccaagacaaagggtgaacattatggaccctgaaaaaataagagaaatatttaccaaatttaatgacttccagaaggtacgtaccaatccactgcttactttgcttgtaagcggccttgttaacctcgaaggagaccgatggagcaaacgtagaaaaatcataaacccttcatttcatcaagataagttgaagaatatgttgccagcattttatcaaagttgttgtgacatgttaagcaaatgggagaagatggtgtgtacagaaggatacagtgagttagatgtgtggccttatcttgtagatttgacaagagatgtgatttcccgatctgcttttggaagcagcttcgaagaaggcagacgaattttccaattactagatgaccaactcgttctcagaatcaaattactacaaactgtttacattccaggatggaggtttttgcccacaaagacaaacagggaggtgaagatgaaacacaaagacataaaagagtcgctcagggaaatgataaaaagaagagagcaggcaattaaagcaggggaagagagcaatgaggacttgttggacatacttgtggaatccaatatcagagaaatggaagcaaagaaaaaggggatgagcattgaggatgtgattgaggaatgcaagctgttttactttgctggccaggagaccacttcggtcttactggtgtggactatggttttattagcaaggtatcccgattggcaaagtaaagcaagagaggaggttttgcatgttttgggtgacagtaaacctgatgccgatggccttaatcgcctcaaagttgtaacaatgatattgtacgaggttctaaggctgtacccatcagcgactgagctaggacgttccgttccaaaagaaataaaactgggaaatttgttgttaccagcaggaacagaaatttcggttccgatcctgctgatccaccatgacaaagatctttggggcgatgacgcacgggaatttaagccagagcggttcgcggaaggggtttccaaagcaacaaagagtcaagtcacgtttctgccattcggatggggtcctaggatctgcattggccagaattttgctatgatggaagccaaaatggcggtggccatgattctccaacgcttctggttcgagctttccccttcctatgctcactctccttgcagcatggtaacgcttcgtccacagcatggtgcacagattattttacataaacttggctctaattga

SEQ ID NO: 33 - protein, 518 aa, Gossypium hirsutum
MEVYHVIRDLVSLLVVGLLMIWGWRALNWVWLAPKRLERCLRQQGFAGNPYRFLSGDIKELSTMSRQTRAKPMPLSDDIGPYVAPFLHQTVNQYGKNSFTWIGPRQRVNIMDPEKIREIFTKFNDFQKVRTNPLLTLLVSGLVNLEGDRWSKRRKIINPSFHQDKLKNMLPAFYQSCCDMLSKWEKMVCTEGYSELDVWPYLVDLTRDVISRSAFGSSFEEGRRIFQLLDDQLVLRIKLLQTVYIPGWRFLPTKTNREVKMKHKDIKESLREMIKRREQAIKAGEESNEDLLDILVESNIREMEAKKKGMSIEDVIEECKLFYFAGQETTSVLLVWTMVLLARYPDWQSKAREEVLHVLGDSKPDADGLNRLKVVTMILYEVLRLYPSATELGRSVPKEIKLGNLLLPAGTEISVPILLIHHDKDLWGDDAREFKPERFAEGVSKATKSQVTFLPFGWGPRICIGQNFAMMEAKMAVAMILQRFWFELSPSYAHSPCSMVTLRPQHGAQIILHKLGSN

SEQ ID NO: 34 - DNA, 50 bp, Amaranthus palmeri
  source 1..50: mol_type = genomic DNA
gcgwrwawwtmaraygkagsaagtaymstskmaykaakkwwgtatrwtgg

SEQ ID NO: 35 - DNA, 47 bp, Amaranthus palmeri
  source 1..47: mol_type = genomic DNA
gaaakccwwcwasamamwmyywmrtwaarksgytaamaatsmwwakk

SEQ ID NO: 36 - DNA, 28 bp, Amaranthus palmeri
  source 1..28: mol_type = genomic DNA
awtygkyrtratwwararmmwaatytac

SEQ ID NO: 37 - DNA, 28 bp, Amaranthus palmeri
  source 1..28: mol_type = genomic DNA
kykatwaatgwktrarawaawryatwgk

SEQ ID NO: 38 - DNA, 21 bp, Amaranthus palmeri
  source 1..21: mol_type = genomic DNA
tawagtwmyatttwtwagtak

SEQ ID NO: 39 - DNA, 14 bp, Amaranthus palmeri
  source 1..14: mol_type = genomic DNA
gatcgggtatgatg

SEQ ID NO: 40 - DNA, 21 bp, Amaranthus palmeri
  source 1..21: mol_type = genomic DNA
actaaatgttaaattttaatc

SEQ ID NO: 41 - DNA, 15 bp, Amaranthus palmeri
  source 1..15: mol_type = genomic DNA
gatcgtattctccta

SEQ ID NO: 42 - DNA, 47 bp, Amaranthus palmeri
  source 1..47: mol_type = genomic DNA
acsysmwmatcatywyymcwwwwtmcyrmtcrmmammwrskattsra

SEQ ID NO: 43 - DNA, 41 bp, Amaranthus palmeri
  source 1..41: mol_type = genomic DNA
aaaktwayatttwragtwgtawymawatayaywwakymttr

SEQ ID NO: 44 - DNA, 29 bp, Amaranthus palmeri
  source 1..29: mol_type = genomic DNA
awavtwawatwvamwmyaawrtwwtttrc

(19) United States
(12) Patent Application Publication
(10) Pub. No.: US 2025/0066807 A1
(43) Pub. Date: Feb. 27, 2025

(54) CYP72A219 GENES CONFERRING HERBICIDE TOLERANCE

(71) Applicants: COLORADO STATE UNIVERSITY RESEARCH FOUNDATION, Fort Collins, CO (US); MONSANTO TECHNOLOGY LLC, St. Louis, MO (US); THE BOARD OF TRUSTEES OF THE UNIVERSITY OF ILLINOIS, Urbana, IL (US)

(72) Inventors: Todd GAINES, Fort Collins, CO (US); Carlos RIGON, Fort Collins, CO (US); Franck DAYAN, Fort Collins, CO (US); Anita Küpper, Kaarst (DE); Roland BEFFA, Liederback am Taunus (DE); Darci GIACOMINI, Denver, CO (US); Patrick TRANEL, St. Joseph, IL (US)

(21) Appl. No.: 18/718,250

(22) PCT Filed: Dec. 14, 2022
(86) PCT No.: PCT/US2022/081561

§ 371 Date: Jun. 10, 2024

Related U.S. Application Data
(60) Provisional application No. 63/289,588, filed on Dec. 14, 2021.

(51) Int. Cl. C12N 15/82; C12Q 1/6895

(52) U.S. Cl. CPC C12N 15/8274; C12N 15/8216; C12Q 1/6895; C12Q 2600/13

(57) ABSTRACT

The present disclosure relates to a plant or plant part comprising increased expression of a polynucleotide encoding a CYP72A219 polypeptide that confers to the plant or plant part tolerance to HPPD inhibitor herbicides, such as tembotrione or mesotrione. The disclosure further provides kits for identifying herbicide resistant plants and methods for determining whether a plant is herbicide resistant.

### CROSS REFERENCE TO RELATED APPLICATIONS

This application claims priority to provisional application U.S. Ser. No. 63/289,588, filed Dec. 14, 2021, which is incorporated herein by reference in its entirety.

### SEQUENCE LISTING XML

The instant application contains a sequence listing, which has been submitted in XML file format by electronic submission and is hereby incorporated by reference in its entirety. Said XML file, created on Dec. 14, 2022, is named P13672WO00.xml and is 79,182 bytes in size.

### TECHNICAL FIELD

The present disclosure relates in general to compositions and methods for conferring plants with tolerance to herbicides.

### BACKGROUND

Weeds left uncontrolled can decrease the yields of several major crops by more than 50% in present North American agronomic systems. Many growers in the United States currently rely heavily on chemical means (i.e., herbicides) to control their weed populations, but the effectiveness of this approach is steadily declining due to growing numbers of herbicide-resistant weeds. While herbicide resistance has been present in the United States since the late 1950s, the widespread adoption of herbicide-tolerant crop varieties in the mid-1990s and overreliance on one or two herbicidal modes of action contributed to an exponential increase in the number of resistant weed species over the last two decades.

Understanding how weeds deal with herbicidal compounds to avoid damage is a major goal of weed science, both to generate workarounds to combat herbicide resistance and to gain insights into plant evolution. Research on herbicide-resistance mechanisms over the last several decades has largely been focused on mutations occurring within genes that encode the target enzymes that are directly inhibited by herbicides (target-site resistance). Only recently has significant progress been made on non-target-site-based resistance (NTSR) mechanisms.

Herbicide tolerant plants are useful in systems in which a plurality of such plants are planted, and can produce a crop, and either prior to planting, or after planting, an herbicide is applied that would otherwise kill or harm the plants but for their tolerance to the herbicide. Undesirable plants are killed or damaged, and the tolerant plants survive. There is a need to produce such plants.

### SUMMARY

Compositions and methods for conferring herbicide tolerance to plants, plant parts, and plant cells are provided. Modified plants having tolerance to an herbicide, the modified plant having increased expression of a polynucleotide encoding a cytochrome P450 72A219 (CYP72A219) polypeptide relative to an unmodified plant are provided. In certain embodiments, the modified plants comprise a heterologous polynucleotide encoding the CYP72A219 polypeptide. Progeny, plant seeds, plant parts, and plant cells of the modified plants are also provided.

Nucleic acid molecules capable of conferring herbicide tolerance comprising a nucleotide sequence selected from: (a) a nucleotide sequence encoding a CYP72A219 polypeptide, wherein the nucleotide sequence has at least 80%, at least 90%, at least 95%, at least 98%, at least 99%, or 100% sequence identity to SEQ ID NO: 1 or 3; or (b) a nucleotide sequence encoding a CYP72A219 polypeptide, wherein the CYP72A219 polypeptide has at least 80%, at least 90%, at least 95%, at least 98%, at least 99%, or 100% sequence identity to SEQ ID NO: 2 or 4 are provided.

Expression cassettes, vectors, biological samples, plants, plant seeds, plant parts, and plant cells comprising the aforementioned nucleic acid molecules are also provided.

CYP72A219 polypeptides comprising an amino acid sequence having at least 80%, at least 90%, at least 95%, at least 98%, at least 99%, or 100% sequence identity to SEQ ID NO: 2 or 4 are provided.

Methods for producing a plant with herbicide tolerance comprising increasing expression of a polynucleotide encoding a CYP72A219 polypeptide in the plant, wherein the herbicide tolerance of the plant is increased when compared to a plant that lacks the increased expression are provided. In certain embodiments, the methods comprise introducing to a plant cell a polynucleotide encoding the CYP72A219 polypeptide, wherein the polynucleotide is operably linked to a heterologous promoter functional in a plant cell; and regenerating a plant from the plant cell.

Methods for controlling undesired vegetation at a plant cultivation site comprising providing at the site a plant that comprises a polynucleotide encoding a CYP72A219 polypeptide, wherein expression of the polynucleotide confers to the plant tolerance to an herbicide; and applying to the site an effective amount of the herbicide are provided.

Methods for controlling the growth of an herbicide resistant weed at a plant cultivation site comprising contacting the weed with a composition that reduces expression or activity of a CYP72A219 polypeptide; and applying to the site an effective amount of the herbicide are provided.

Commodity plant products prepared from the aforementioned plants, plant parts, and plant cells are provided. In certain embodiments, the product comprises the CYP72A219 polypeptide or the polynucleotide encoding the CYP72A219 polypeptide. Methods for producing a commodity plant product comprising processing the aforementioned plants or plant parts to obtain the product are also provided.

Methods for identifying an herbicide-resistant plant are provided. In certain embodiments, the methods comprise obtaining a sample from a plant suspected of having herbicide resistance; quantifying expression of a CYP72A219 gene in the sample; and determining that the plant is herbicide-resistant based on the quantification. In certain other embodiments, the methods comprise obtaining a sample from a plant suspected of having herbicide resistance; detecting one or more motifs in the promoter of a CYP72A219 gene in the sample; and determining that the plant is herbicide-resistant based on the presence of the one or more motifs.

Kits for identifying an herbicide-resistant plant comprising at least two primers, wherein the at least two primers recognize a CYP72A219 gene that is differentially expressed in an herbicide-resistant plant compared to an herbicide-sensitive plant of the same species are also provided.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### BRIEF DESCRIPTION OF THE FIGURES

The following drawings form part of the specification and are included to further demonstrate certain embodiments or various aspects of the invention. In some instances, embodiments of the invention can be best understood by referring to the accompanying figures in combination with the detailed description presented herein. The description and accompanying figures may highlight a certain specific example, or a certain aspect of the invention. However, one skilled in the art will understand that portions of the example or aspect may be used in combination with other examples or aspects of the invention.

FIG. 1A-B shows gene expression analysis of cytochrome P450 genes. FIG. 1A shows four cytochrome P450 genes were differentially expressed between NER and NES: MAKER_25717 [CYP72A219 (a), chromosome 4]; MAKER_25718 [CYP72A219 (b), chromosome 4]; MAKER_29886 [CYP72A219 (c), chromosome 16]; and MAKER_10107 [CYP81E, chromosome 4]. FIG. 1B shows expression of all four genes was induced in NES at 6 and 12 hours after treatment (HAT) with tembotrione.

FIG. 2A-B shows motifs in the promoter of the CYP72A219 gene. The promoter sequences in susceptible individuals (FIG. 2A) and resistant individuals (FIG. 2B) have uniquely different motifs: motif 8 (SEQ ID NO: 34), motif 11 (SEQ ID NO: 35), motif 16 (SEQ ID NO: 36), motif 17 (SEQ ID NO: 37), motif 19 (SEQ ID NO: 38), motif 20 (SEQ ID NO: 39), motif 22 (SEQ ID NO: 40), and motif 24 (SEQ ID NO: 41) are found only in resistant individuals; motif 13 (SEQ ID NO: 42), motif 14 (SEQ ID NO: 43), and motif 15 (SEQ ID NO: 44) are found only in susceptible individuals.

FIG. 3A-F shows tembotrione metabolism in a heterologous yeast expression system. The CYP72A219 gene produced the same hydroxy-tembotrione metabolite as produced in planta. Negative controls showed that the yeast line did not detoxify tembotrione and a positive control gene produced hydroxy-tembotrione as expected.

FIG. 4A-D shows CYP72A219 expression in different HPPD-resistant Palmer amaranth populations. FIG. 4A shows plant survival in a tembotrione dose response for 11 Palmer amaranth populations from surveys conducted in 2019 and 2013, compared to the known resistant population collected in 2012 (AMAPA USA 12001) and the known sensitive population. FIG. 4B-D show relative gene expression of genes CYP72A219 (a) (FIG. 4B), a CYP not involved in herbicide metabolism (FIG. 4C), and CYP81E (FIG. 4D), measured in 11 tembotrione resistant populations, 1 reference resistant population, and 2 sensitive resistant populations, both before and 6 h after tembotrione treatment. Numbers above data points indicate fold change relative to HPPD S untreated.

### BRIEF DESCRIPTION OF THE SEQUENCES

SEQ ID NO: 1 is the nucleotide sequence encoding CYP72A219 (a) of chromosome 4 from Amaranthus palmeri.

SEQ ID NO: 2 is the amino acid sequence encoded by SEQ ID NO: 1.

SEQ ID NO: 3 is the nucleotide sequence encoding an allelic variant of CYP72A219 (a) of chromosome 4 from Amaranthus palmeri.

SEQ ID NO: 4 is the amino acid sequence encoded by SEQ ID NO: 3.

SEQ ID NO: 5 is the nucleotide sequence encoding CYP72A219 (b) of chromosome 4 from Amaranthus palmeri.

SEQ ID NO: 6 is the amino acid sequence encoded by SEQ ID NO: 5.

SEQ ID NO: 7 is the nucleotide sequence encoding CYP72A219 (c) of chromosome 16 from Amaranthus palmeri.

SEQ ID NO: 8 is the amino acid sequence encoded by SEQ ID NO: 7.

SEQ ID NO: 9 is the nucleotide sequence encoding CYP81E of chromosome 4 from Amaranthus palmeri.

SEQ ID NO: 10 is the amino acid sequence encoded by SEQ ID NO: 9.

SEQ ID NO: 11 through SEQ ID NO: 15 are promoter sequences of CYP72A219 (a) from resistant plants.

SEQ ID NO: 16 through SEQ ID NO: 19 are promoter sequences of CYP72A219 (a) from susceptible plants.

SEQ ID NOs: 20, 22, 24, 26, 28, 30, and 32 are nucleotide sequences of orthologs of SEQ ID NO: 1.

SEQ ID NOs: 21, 23, 25, 27, 29, 31, and 33 are the amino acid sequences encoded by SEQ ID NOs: 20, 22, 24, 26, 28, 30, and 32, respectively.

SEQ ID NO: 34 through SEQ ID NO: 41 are promoter sequence motifs unique to resistant plants.

SEQ ID NO: 42 through SEQ ID NO: 44 are promoter sequence motifs unique to susceptible plants.

### DETAILED DESCRIPTION

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation; the preferred materials and methods are described herein. In describing and claiming the embodiments of the present invention, the following terminology will be used in accordance with the definitions set out below.

It is to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about”, as used herein, refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

As used herein, the phrase “biological sample” refers to either intact or non-intact (e.g., milled seed or plant tissue, chopped plant tissue, lyophilized tissue) plant tissue. It may also be an extract comprising intact or non-intact seed or plant tissue. The biological sample can comprise flour, meal, syrup, oil, starch, and cereals manufactured in whole or in part to contain crop plant by-products. In certain embodiments, the biological sample is “non-regenerable” (i.e., incapable of being regenerated into a plant or plant part).

As used herein, the term “confer” refers to providing a characteristic or trait, such as herbicide tolerance or resistance and/or other desirable traits to a plant.

The term “control of undesired vegetation or weeds” is to be understood as meaning the killing of weeds and/or otherwise retarding or inhibiting the normal growth of the weeds. Weeds, in the broadest sense, are understood as meaning all those plants which grow in locations where they are undesired. The weeds of the present disclosure include, for example, dicotyledonous and monocotyledonous weeds. Dicotyledonous weeds include, but are not limited to, weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera. In addition, the weeds of the present disclosure can include, for example, crop plants that are growing in an undesired location. For example, a volunteer maize plant that is in a field that predominantly comprises soybean plants can be considered a weed, if the maize plant is undesired in the field of soybean plants.

As used herein, the term “DNA” or “DNA molecule” refers to a double-stranded DNA molecule of genomic or synthetic origin, i.e. a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule.

As used herein, an “endogenous gene” or a “native copy” of a gene refers to a gene that originates from within a given organism, cell, tissue, genome, or chromosome. An “endogenous gene” or a “native copy” of a gene is a gene that was not previously modified by human action. Similarly, an “endogenous protein” refers to a protein encoded by an endogenous gene.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present disclosure. Typically, the effective amount of an herbicide is an amount that is routinely used in agricultural production systems to kill weeds of interest. Such an amount for a given herbicide is known to those of ordinary skill in the art. Herbicidal activity is exhibited by herbicides useful for the present disclosure when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, the herbicide treatments can be applied PPI (Pre Plant Incorporated), PPSA (Post plant surface applied), PRE- or POST-emergent. Postemergent treatment typically occurs to relatively immature undesirable vegetation to achieve the maximum control of weeds.

By a “herbicide-tolerant” or “herbicide-resistant” plant, it is intended that a plant that is tolerant or resistant to at least one herbicide at a level that would normally kill, or inhibit the growth of, a normal or wildtype plant. Levels of herbicide that normally inhibit growth of a non-tolerant plant are known and readily determined by those skilled in the art. Examples include the amounts recommended by manufacturers for application. The maximum rate is an example of an amount of herbicide that would normally inhibit growth of a non-tolerant plant. For the present disclosure, the terms “herbicide-tolerant” and “herbicide-resistant” are used interchangeably and are intended to have an equivalent meaning and an equivalent scope. Similarly, the terms “herbicide-tolerance” and “herbicide-resistance” are used interchangeably and are intended to have an equivalent meaning and an equivalent scope. Similarly, the terms “tolerant” and “resistant” are used interchangeably and are intended to have an equivalent meaning and an equivalent scope. As used herein, in regard to an herbicidal composition useful in various embodiments hereof, terms such as herbicides, and the like, refer to those agronomically acceptable herbicide active ingredients (A.I.) recognized in the art. As used herein, an “herbicide tolerance trait” is a trait imparting improved herbicide tolerance to a plant as compared to the wild-type plant.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In one aspect, a modified plant provided herein comprises no non-plant genetic material or sequences. In yet another aspect, a modified plant provided herein comprises no interspecies genetic material or sequences.

As used herein, “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A progeny plant can be from any filial generation, e.g., F1, F2, F3, F4, F5, F6, F7, etc. A plant cell is a biological cell of a plant, taken from a plant or derived through culture from a cell taken from a plant. Plant parts include, but are not limited to seeds microspores, pollen, anthers, silk, spike, ovules, ovaries, flowers, pods, cobs, embryos, stems, leaves, roots, and calli.

The term “polynucleotide” as used herein is a nucleic acid molecule comprising a plurality of polymerized nucleotides, e.g., at least about five consecutive polymerized nucleotides. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like. The polynucleotide can be single-stranded or double-stranded DNA or RNA. The polynucleotide optionally comprises modified bases or a modified backbone. The polynucleotide can be, e.g., genomic DNA or RNA, a transcript (such as an mRNA), a cDNA, a PCR product, a cloned DNA, a synthetic DNA or RNA, or the like. The polynucleotide can be combined with carbohydrate, lipids, protein, or other materials to perform a particular activity such as transformation or form a useful composition such as a peptide nucleic acid (PNA). The polynucleotide can comprise a sequence in either sense or antisense orientations. “Oligonucleotide” is substantially equivalent to the terms amplimer, amplicon, primer, oligomer, element, target, and probe and in some embodiments is single-stranded.

The term “primer” as used herein encompasses any nucleic acid that is capable of priming the synthesis of a nascent nucleic acid in a template-dependent process, such as PCR. Typically, primers are oligonucleotides from 10 to 30 nucleotides in length, but longer sequences may be used. Primers may be provided in single or double-stranded form. Probes may be used as primers, but are designed to bind to the target DNA or RNA and need not be used in an amplification process.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Illustrative plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters that initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter that is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter that is active under most environmental conditions.

As used herein, “recombinant,” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if that nucleotide sequence has been removed from its natural context and cloned into any type of artificial nucleic acid vector. The term recombinant also can refer to an organism having a recombinant material, e.g., a plant that comprises a recombinant nucleic acid can be considered a recombinant plant.

“Regulatory elements” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory elements may include, but are not limited to, promoters, translation leader sequences, introns, and polyadenylation recognition sequences. Regulatory elements present on a recombinant DNA construct that is introduced into a cell can be endogenous to the cell, or they can be heterologous with respect to the cell. The terms “regulatory element” and “regulatory sequence” are used interchangeably herein.

A “sequence” means a sequential arrangement of nucleotides or amino acids. The boundaries of a protein-coding sequence may be determined by a translation start codon at the 5′-terminus and a translation stop codon at the 3′-terminus. In some embodiments, a protein-coding molecule may comprise a DNA sequence encoding a protein sequence. In some embodiments, a protein-coding molecule may comprise a RNA sequence encoding a protein sequence. As used herein, “transgene expression”, “expressing a transgene”, “protein expression”, and “expressing a protein” mean the production of a protein through the process of transcribing a DNA molecule into messenger RNA (mRNA) and translating the mRNA into polypeptide chains, which are ultimately folded into proteins.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., Madison, Wis.), and MUSCLE (version 3.6) (Edgar, “MUSCLE: multiple sequence alignment with high accuracy and high throughput” Nucleic Acids Research 32 (5): 1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

As used herein, “vector” includes reference to a nucleic acid used in transfection of a host cell and into which can be inserted a polynucleotide. Vectors are often replicons. Expression vectors permit transcription of a nucleic acid inserted therein.

### CYP72A219 Polynucleotides and Polypeptides

Cytochrome P450 72A219 (CYP72A219) sequences are provided that confer herbicide tolerance. Such sequences include the amino acid sequences set forth in SEQ ID NO: 2 and 4, and variants thereof. Also provided are polynucleotide sequences encoding such amino acid sequences, including SEQ ID NO: 1 and 3.

According to several embodiments crop plants are transformed with a gene encoding a CYP72A219 polypeptide capable of inactivating certain 4-Hydroxyphenylpyruvate dioxygenase (HPPD) inhibitor herbicides and also, optionally, other types of herbicides.

Additional polynucleotide sequences encoding a CYP72A219 polypeptide may be identified using methods well known in the art based on their ability to confer tolerance to an herbicide of interest. For example, candidate CYP72A219 genes are transformed into and expressed in suitable yeast strains and selected on the basis of their ability to oxidize test herbicides in vitro (Siminszky et al (1999) PNAS (USA) 96:1750-1755). Suitable yeast strains include WAT11 or WAT21 which also comprise a suitable plant cytochrome P450 competent reductase. Following induction for a suitable period (for example, depending on the inducible promoter used in the transformation vector, with galactose) cells are grown up, harvested, broken, the microsome fraction prepared by the usual means and assayed with NADPH for the ability to oxidize ¹⁴C-labeled herbicide. Optionally, assays are carried out using whole cells in culture.

Alternatively, candidate CYP72A219 genes are expressed in tobacco, Arabidopsis, or other easily transformed, herbicide sensitive plant and the resultant transformant plants assessed for their tolerance to HPPD inhibitor herbicide(s) or other herbicides of interest. Optionally the plants, or tissue samples taken from plants, are treated with herbicide and assayed in order to assess the rate of metabolic conversion of parent herbicide to oxidized metabolic degradation products.

Those skilled in the art may also find further candidate CYP72A219 genes based on genome synteny and sequence similarity. In one embodiment, additional gene candidates can be obtained by hybridization or PCR using sequences based on the CYP72A219 nucleotide sequences noted above.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art. See, for example, Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York).

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

By “hybridizing to” or “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point (Tₘ) for the specific sequence at a defined ionic strength and pH. Typically, under “stringent conditions” a probe will hybridize to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

The following are examples of sets of hybridization/wash conditions that may be used to clone nucleotide sequences that are homologues of reference nucleotide sequences: a reference nucleotide sequence preferably hybridizes to the reference nucleotide sequence in 7% sodium dodecyl sulfate (SDS), 0.5 M NaPO₄, 1 mM EDTA at 50° C. with washing in 2×SSC, 0.1% SDS at 50° C., more desirably in 7% sodium dodecyl sulfate (SDS), 0.5 M NaPO₄, 1 mM EDTA at 50° C. with washing in 1×SSC, 0.1% SDS at 50° C., more desirably still in 7% sodium dodecyl sulfate (SDS), 0.5 M NaPO₄, 1 mM EDTA at 50° C. with washing in 0.5×SSC, 0.1% SDS at 50° C., preferably in 7% sodium dodecyl sulfate (SDS), 0.5 M NaPO₄, 1 mM EDTA at 50° C. with washing in 0.1×SSC, 0.1% SDS at 50° C., more preferably in 7% sodium dodecyl sulfate (SDS), 0.5 M NaPO₄, 1 mM EDTA at 50° C. with washing in 0.1×SSC, 0.1% SDS at 65° C.

Several embodiments also relate to the use of CYP72A219 or variants thereof that confer tolerance to herbicides, including HPPD inhibitor herbicides. “Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode CYP72A219 polypeptides described above. Naturally occurring allelic variants can be identified with the use of well-known molecular biology techniques, for example, with polymerase chain reaction (PCR) and hybridization techniques as outlined above. Variant polynucleotides also include synthetically derived polynucleotides, such as those generated, for example, by using site-directed mutagenesis but which still encode a CYP72A219 polypeptide conferring herbicide tolerance. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide.

Variants of a particular polynucleotide encoding a CYP72A219 that confers herbicide tolerance are encompassed and can be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and algorithms described herein. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

Methods of alignment of sequences for comparison are well known in the art and can be accomplished using mathematical algorithms such as the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; and the algorithm of Karlin and Altschul (1990) Proc. Natl. Acad. Sci. U.S. Pat. No. 872,264, modified as in Karlin and Altschul (1993) Proc. Natl. Acad. Sci. USA 90:5873-5877. Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA).

Several embodiments relate to increasing expression of a CYP72A219 gene in a plant. The term “increased expression” or “overexpression” as used herein means any form of expression that is additional to the original wild-type expression level. The original wild-type expression level might also be zero (absence of expression). Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a nucleic acid encoding the protein of interest. For example, endogenous promoters may be altered in vivo by mutation, deletion, and/or substitution (see, Kmiec, U.S. Pat. No. 5,565,350; Zarling et al., WO9322443), or isolated promoters may be introduced into a plant cell in the proper orientation and distance from a CYP72A219 gene so as to control the expression of the gene.

Targeted modification of plant genomes through the use of genome editing methods can be used to increase expression of a CYP72A219 gene through modification of plant genomic DNA. Genome editing methods can enable targeted insertion of one or more nucleic acids of interest into a plant genome. Examples methods for introducing donor polynucleotides into a plant genome or modifying genomic DNA of a plant include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas system, such as a CRISPR/Cas9 system, a CRISPR/Cpf1 system, a CRISPR/CasX system, a CRISPR/CasY system, or a CRISPR/Cascade system). Methods of genome editing to modify, delete, or insert nucleic acid sequences into genomic DNA are known in the art.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the disclosure as described herein.

With regard to a defined polypeptide, it will be appreciated that % identity figures higher than those provided above will encompass preferred embodiments. Thus, where applicable, in light of the minimum % identity figures, it is preferred that the CYP72A219 polypeptide comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to SEQ ID NO: 2 or 4.

By “variant” polypeptide is intended a polypeptide derived from the protein of SEQ ID NO: 2 or 4, by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or from human manipulation. Methods for such manipulations are generally known in the art.

“Derivatives” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. Thus, functional variants and fragments of the CYP72A219 polypeptides, and nucleic acid molecules encoding them, also are within the scope of the present disclosure, and unless specifically described otherwise, irrespective of the origin of said polypeptide and irrespective of whether it occurs naturally.

In addition, one of ordinary skill in the art will further appreciate that changes can be introduced by mutation into the nucleotide sequences thereby leading to changes in the amino acid sequence of the encoded proteins without altering the biological activity of the proteins. Thus, for example, an isolated polynucleotide molecule encoding a CYP72A219 polypeptide having an amino acid sequence that differs from that of SEQ ID NO: 2 or 4 can be created by introducing one or more nucleotide substitutions, additions, or deletions into the corresponding nucleotide sequence, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present disclosure. For example, preferably, conservative amino acid substitutions may be made at one or more predicted preferably nonessential amino acid residues. A “nonessential” amino acid residue is a residue that can be altered from the wild-type sequence of a protein without altering the biological activity, whereas an “essential” amino acid residue is required for biological activity.

A deletion refers to removal of one or more amino acids from a protein. An insertion refers to one or more amino acid residues being introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag·100 epitope, c-myc epitope, FLAG®-epitope, lacZ, CMP (calmodulin-binding peptide), HA epitope, protein C epitope VSV epitope, and fluorescent tags such a green fluorescent protein (GFP) or yellow fluorescent protein (YFP).

A substitution refers to replacement of amino acids of the protein with other amino acids having similar properties (such as similar hydrophobicity, hydrophilicity, antigenicity, propensity to form or break α-helical structures or β-sheet structures). Amino acid substitutions are typically of single residues but may be clustered depending upon functional constraints placed upon the polypeptide and may range from 1 to 10 amino acids; insertions will usually be of the order of about 1 to 10 amino acid residues. A conservative amino acid substitution is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such substitutions would not be made for conserved amino acid residues, or for amino acid residues residing within a conserved motif. Conservative substitution tables are well known in the art (see for example Creighton (1984) Proteins. W.H. Freeman and Company (Eds).

Amino acid substitutions, deletions and/or insertions may readily be made using peptide synthetic techniques well known in the art, such as solid phase peptide synthesis and the like, or by recombinant DNA manipulation. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site Directed mutagenesis (Stratagene, San Diego, Calif.), PCR-mediated site-directed mutagenesis or other site-directed mutagenesis protocols.

In certain embodiments, the polypeptides include at least one amino acid substitution, insertion, or deletion so that they do not recite a naturally occurring amino acid sequence.

Orthologs” and “paralogs” encompass evolutionary concepts used to describe the ancestral relationships of genes. Paralogs are genes within the same species that have originated through duplication of an ancestral gene; orthologs are genes from different organisms that have originated through speciation, and are also derived from a common ancestral gene. Orthologs and paralogs of SEQ ID NO: 1 or 3 encompassed by the present disclosure include, but are not limited to, polynucleotides encoding SEQ ID NO: 21, 23, 25, 27, 29, 31, or 33.

TABLE 1

| | | Coding | Amino acid |
|---|---|---|---|
| Gene | Species | sequence | sequence |
| AH007959 | Amaranthus | SEQ ID NO: 20 | SEQ ID NO: 21 |
| | hypochondriacus | | |
| Bevul.4G087600 | Beta vulgaris | SEQ ID NO: 22 | SEQ ID NO: 23 |
| Spov3_chr1.04887 | Spinacia oleracea | SEQ ID NO: 24 | SEQ ID NO: 25 |
| AUR62019700-RA | Chenopodium quinoa | SEQ ID NO: 26 | SEQ ID NO: 27 |
| Soltu.DM.07G021190 | Solanum tuberosum | SEQ ID NO: 28 | SEQ ID NO: 29 |
| Solyc07g055350 | Solanum lycopersicum | SEQ ID NO: 30 | SEQ ID NO: 31 |
| Gohir.D01G035400 | Gossypium hirsutum | SEQ ID NO: 32 | SEQ ID NO: 33 |

### HPPD Inhibitor Herbicides and Other Herbcides

The CYP72A219 polypeptides of the disclosure are capable of inactivating one or more herbicides, including 4-hydroxyphenylpyruvate dioxygenase (HPPD) inhibitor herbicides. HPPDs are enzymes which catalyze the reaction in which para-hydroxyphenylpyruvate, a tyrosine degradation product, is transformed into homogentisate, the precursor in plants of tocopherol and plastoquinone (Crouch N. P. et al. (1997), Tetrahedron, 53, 20, 6993-7010, Fritze et al. (2004), Plant Physiology 134:1388-1400). Tocopherol acts as a membrane-associated antioxidant. Plastoquinone, firstly acts as an electron carrier between PSII and the cytochrome b6/f complex and secondly, is a redox cofactor for phytoene desaturase, which is involved in the biosynthesis of carotenoids.

Inhibition of HPPD leads to uncoupling of photosynthesis, deficiency in accessory light-harvesting pigments and, most importantly, to destruction of chlorophyll by UV-radiation and reactive oxygen species (bleaching) due to the lack of photo protection normally provided by carotenoids (Norris et al. (1995), Plant Cell 7:2139-2149). Bleaching of photosynthetically active tissues leads to growth inhibition and plant death.

HPPD inhibitor herbicides include triketones, isoxazoles, and pyrazolinates.

The triketones include, but are not limited to, sulcotrione [i.e. 2-[2-chloro-4-(methylsulfonyl)benzoyl]-1,3-cyclohexanedione], mesotrione [i.e. 2-[4-(methylsulfonyl)-2-nitrobenzoyl]-1,3-cyclohexanedione]; tembotrione [i.e. 2-[2-chloro-4-(methylsulfonyl)-3-[(2,2,2,-tri-fluoroethoxy)methyl]benzoyl]-1,3-cyclo-hexanedione]; tefuryltrione [i.e. 2-[2-chloro-4-(methylsulfonyl)-3-[[(tetrahydro-2-furanyl)methoxy]methyl]benzoyl]-1,3 cyclohexanedione]]; bicyclopyrone [i.e. 4-hydroxy-3-[[2-[(2-methoxyethoxy)methyl]-6-(trifluoromethyl)-3-pyridinyl]carbonyl]bicyclo[3.2.1]oct-3-en-2-one]; benzobicyclone [i.e. 3-(2-chloro-4-mesylbenzoyl)-2-phenylthiobicyclo[3.2.1]oct-2-en-4-one];

The isoxazoles include, but are not limited to isoxaflutole [i.e. (5-cyclopropyl-4-isoxazolyl) [2-(methylsulfonyl)-4-(trifluoromethyl)phenyl]methanone] or corresponding diketonitriles.

The pyrazolinones include, but are not limited to, topramezone [i.e. [3-(4,5-dihydro-3-isoxazolyl)-2-methyl-4-(methylsulfonyl)phenyl](5-hydroxy-1-methyl-1H-pyrazol-4-yl)methanone], pyrasulfotole [(5-hydroxy-1,3-dimethylpyrazol-4-yl(2-mesyl-4trifluaromethylphenyl)methanone]; pyrazoxyfen[2-[4-(2,4-dichlorobenzoyl)-1,3-dimethylpyrazol-5-yloxy]acetophenone].

Further compound classes of HPPD inhibitor herbicides are the N-(tetrazol-5-yl)- and N-(triazol-5-yl)arylcarboxamides as disclosed in PCT/EP2011/064820 and the N-(1,2,5-Oxadiazol-3-yl)benzamides as disclosed in WO2011/035874.

The CYP72A219 polypeptides of the disclosure may also be capable of inactivating one or more other types of herbicides including, for example, acetolactate synthase (ALS) inhibitor herbicides, auxin herbicides, protoporphyrinogen oxidase (PPO) inhibitor herbicides, acetyl-CoA carboxylase (ACCase) inhibitor herbicides, photosystem II (PSII) inhibitor herbicides, or phytoene desaturase (PDS) inhibitor herbicides.

Examples of ALS inhibitor herbicides include, but are not limited to, sulfonylureas (such as bensulfuron, chlorsulfuron, halosulfuron, nicosulfuron, rimsulfuron, sulfometuron, and triflusulfuron); imidazolinones (such as imazamox, imazethapyr, imazapic, imazamethabenz-methyl, and imazaquin); triazolopyrimidines (such as flumetsulam, diclosulam, florasulam, chloransulam-methyl, and metosulam); and triazolinones (such as flucarbazone, thiencarbazone-methyl, and propoxycarbazone).

Examples of auxin herbicides include benzoic acids, phenoxycarboxylic acids, pyridine carboxylic acids, quinoline carboxylic acids, semi-carbasones, diflufenzopyr, 2,4-D, 2,4-DB, MCPA, MCPB, mecoprop, dicamba, clopyralid, fluroxypyr, picloram, triclopyr, aminopyralid, aminocyclopyrachlor, and quinclorac.

Examples of ACCase inhibitor herbicides include aryloxyphenoxypropionates (such as clodinafop, cyhalofop, diclofop, fenoxaprop, fluazifop, haloxyfop, propaquizafop, and quizalofop); cyclohexanediones (such as alloxydim, butroxydim, clethodim, cycloxydim, profoxydim, sethoxydim, tepraloxydim, and tralkoxydim), and phenylpyrazolines (such as pinoxaden).

Examples of PPO inhibitor herbicides include, but are not limited to, diphenylethers (such as acifluorfen, aclonifen, bifenox, ethoxyfen, fluoronitrofen, furyloxyfen, halosafen, chlomethoxyfen, fluoroglycofen, lactofen, oxyfluorfen, and fomesafen); thiadiazoles (such as fluthiacet-methyl and thidiazimin); pyrimidinediones or phenyluracils (such as benzfendizone, butafenacil, ethyl[3-2-chloro-4-fluoro-5-(1-methyl-6-trifluoromethyl-2,4-dioxo-1,2,3,4-tetrahydropyrimidin-3-yl)phenoxy]-2-pyridyloxy]acetate, flupropacil, saflufenacil, and tiafenacil); phenylpyrazoles (such as fluazolate, pyraflufen and pyraflufen-ethyl); oxadiazoles (such as oxadiargyl and oxadiazon); triazolinones (such as azafenidin, bencarbazone, carfentrazone, and sulfentrazone); oxazolidinediones (such as pentoxazone); N-phenylphthalimides (such as cinidon-ethyl, flumiclorac, flumiclorac-pentyl, and flumioxazin); benzoxazinone derivatives (such as 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione); flufenpyr and flufenpyr-ethyl; pyraclonil; and profluazol.

Examples of PSII inhibitor herbicides include, but are not limited to, bentazon, linuron, hexazinone, metribuzin, atrazine, diuron, isoproturon, monolinuron, desmedipham, metamitron, propanil, amicarbzone, fluometuron, phenmedipham, pyridate, ametryn, cynazine, dimefuron, fluometuron, methibenzuron, metoxuron, prometryn, simazine, simetryn, terbacil, terbuthylazine, chlorotoluron, and trietazine.

Examples of PDS inhibitor herbicides include, but are not limited to, norflurazon, diflufenican, fluorochloridone, flurtamone, picolinafen, and fluridone.

HPPD inhibitor herbicides or other herbicides may be applied to a plant growth area comprising the plants and seeds provided by the compositions and methods described herein as a method for controlling weeds. Plants and seeds provided by the compositions and methods described herein comprise an herbicide tolerance trait and as such are tolerant to the application of one or more herbicides, including HPPD inhibitor herbicides. The herbicide application may be the recommended commercial rate (1×) or any fraction or multiple thereof, such as twice the recommended commercial rate (2×). Herbicide rates may be expressed as acid equivalent per pound per acre (lb ae/acre) or acid equivalent per gram per hectare (g ae/ha) or as pounds active ingredient per acre (lb ai/acre) or grams active ingredient per hectare (g ai/ha), depending on the herbicide and the formulation. The plant growth area may or may not comprise weed plants at the time of herbicide application.

Herbicide applications may be sequentially or tank mixed with one, two, or a combination of several herbicides or any other compatible herbicide. Multiple applications of one herbicide or of two or more herbicides, in combination or alone, may be used over a growing season to areas comprising plants expressing CYP72A219 protein as described herein for the control of a broad spectrum of dicot weeds, monocot weeds, or both, for example, two applications (such as a pre-planting application and a post-emergence application or a pre-emergence application and a post-emergence application) or three applications (such as a pre-planting application, a pre-emergence application, and a post-emergence application or a pre-emergence application and two post-emergence applications).

### Expression Constructs

Polynucleotides as described herein can be provided in an expression construct. Expression constructs generally include regulatory elements that are functional in the intended host cell in which the expression construct is to be expressed. Thus, a person of ordinary skill in the art can select regulatory elements for use in bacterial host cells, yeast host cells, plant host cells, insect host cells, and mammalian host cells. Regulatory elements include promoters, transcription termination sequences, translation termination sequences, enhancers, and polyadenylation elements. As used herein, the term “expression construct” refers to a combination of nucleic acid sequences that provides for transcription of an operably linked nucleic acid sequence. As used herein, “operably linked” means two DNA molecules linked in manner so that one may affect the function of the other. Operably-linked DNA molecules may be part of a single contiguous molecule and may or may not be adjacent. For example, a promoter is operably linked with a polypeptide-encoding DNA molecule in a DNA construct where the two DNA molecules are so arranged that the promoter may affect the expression of the DNA molecule.

As used herein, the term “heterologous” refers to the relationship between two or more items derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell, seed, or organism into which it is inserted when it would not naturally occur in that particular cell, seed, or organism.

An expression construct can comprise a promoter sequence operably linked to a polynucleotide sequence encoding a CYP72A219 polypeptide as described herein. Promoters can be incorporated into a polynucleotide using standard techniques known in the art. Multiple copies of promoters or multiple promoters can be used in an expression construct as described herein. In some embodiments, a promoter can be positioned about the same distance from the transcription start site in the expression construct as it is from the transcription start site in its natural genetic environment. Some variation in this distance is permitted without a substantial decrease in promoter activity. A transcription start site is typically included in the expression construct.

Embodiments relate to a recombinant DNA molecule encoding a CYP72A219 polypeptide, wherein the recombinant DNA molecule is further defined as operably linked to a heterologous regulatory element. In specific embodiments, the heterologous regulatory element is a promoter functional in a plant cell. In further embodiments, the promoter is an inducible promoter.

If the expression construct is to be provided in or introduced into a plant cell, then plant viral promoters, such as, for example, a cauliflower mosaic virus (CaMV) 35S (including the enhanced CaMV 35S promoter (see, for example U.S. Pat. No. 5,106,739)) or a CaMV 19S promoter or a cassava vein mosaic can be used. Other promoters that can be used for expression constructs in plants include, for example, zein promoters including maize zein promoters, prolifera promoter, Ap3 promoter, heat shock promoters, T-DNA 1′- or 2′-promoter of A. tumefaciens, polygalacturonase promoter, chalcone synthase A (CHS-A) promoter from petunia, tobacco PR-la promoter, ubiquitin promoter, actin promoter, alcA gene promoter, pin2 promoter (Xu et al., 1993), maize Wipl promoter, maize trpA gene promoter (U.S. Pat. No. 5,625,136), maize CDPK gene promoter, and RUBISCO SSU promoter (U.S. Pat. No. 5,034,322) can also be used. Constitutive promoters (such as the CaMV, ubiquitin, actin, or NOS promoter), developmentally regulated promoters, and inducible promoters (such as those promoters than can be induced by heat, light, hormones, or chemicals) are also contemplated for use with polynucleotide expression constructs described herein.

Expression constructs may optionally contain a transcription termination sequence, a translation termination sequence, a sequence encoding a signal peptide, and/or enhancer elements. Transcription termination regions can typically be obtained from the 3′ untranslated region of a eukaryotic or viral gene sequence. Transcription termination sequences can be positioned downstream of a coding sequence to provide for efficient termination. A signal peptide sequence is a short amino acid sequence typically present at the amino terminus of a protein that is responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of an operably linked signal peptide sequence is contemplated for use with the polypeptides described herein. Classical enhancers are cis-acting elements that increase gene transcription and can also be included in the expression construct. Classical enhancer elements are known in the art, and include, but are not limited to, the CaMV 35S enhancer element, cytomegalovirus (CMV) early promoter enhancer element, and the SV40 enhancer element. Intron-mediated enhancer elements that enhance gene expression are also known in the art. These elements must be present within the transcribed region and are orientation dependent. Examples include the maize shrunken-1 enhancer element (Clancy and Hannah, 2002).

Optionally the gene encoding the CYP72A219 polypeptide is codon-optimized to remove features inimical to expression and codon usage is optimized for expression in the particular crop (see, for example, U.S. Pat. No. 6,051,760; EP 0359472; EP 80385962; EP 0431829; and Perlak et al. (1991) PNAS USA 88:3324-3328; all of which are herein incorporated by reference).

In certain embodiments, the nucleic acid molecules include at least one nucleotide substitution, insertion, or deletion so that they do not recite a naturally occurring nucleic acid sequence.

### Transformation Methods

Several embodiments relate to plant cells, plant tissues, plants, and seeds that comprise a recombinant DNA as described herein. In some embodiments, cells, tissues, plants, and seeds comprising the recombinant DNA molecules exhibit tolerance to HPPD inhibitor herbicide.

Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are Agrobacterium-mediated transformation and microprojectile bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, which is incorporated herein by reference in its entirety. Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants.

In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers which confer resistance to a selective agent, such as an antibiotic or an herbicide. Any of the herbicides to which plants of this disclosure can be resistant is an agent for selective markers. Potentially transformed cells are exposed to the selective agent. In the population of surviving cells are those cells where, generally, the resistance-conferring gene is integrated and expressed at sufficient levels to permit cell survival. Cells can be tested further to confirm stable integration of the exogenous DNA. Commonly used selective marker genes include those conferring resistance to antibiotics such as kanamycin and paromomycin (nptII), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers which provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plants with Herbicide Tolerance

Several embodiments relate to plant cells, plant tissues, plants, and seeds that comprise a polynucleotide encoding a CYP72A219 polypeptide, wherein expression of the polynucleotide confers tolerance to an herbicide. Plants may be monocots or dicots, and may include, for example, rice, wheat, barley, oats, rye, sorghum, maize, grape, tomato, potato, lettuce, broccoli, cucumber, peanut, melon, pepper, carrot, squash, onion, soybean, alfalfa, sunflower, cotton, canola, sugar cane, and sugar beet plants.

Plants that are particularly useful in the methods of the present disclosure include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. In certain embodiments, the plant is a crop plant. Examples of crop plants include inter alia soybean, sunflower, canola, alfalfa, rapeseed, cotton, tomato, potato, or tobacco.

Also provided are a progeny or a descendant of an herbicide-tolerant plant as well as seeds derived from the herbicide-tolerant plants and cells derived from the herbicide-tolerant plants as described herein.

The present disclosure also provides a progeny or descendant plant derived from a plant comprising in at least some of its cells a polynucleotide operably linked to a promoter functional in a plant cell, the promoter capable of expressing a CYP72A219 polypeptide encoded by the polynucleotide, wherein the progeny or descendant plant comprises in at least some of its cells the recombinant polynucleotide operably linked to the promoter, the expression of the CYP72A219 polypeptide conferring to the progeny or descendant plant tolerance to the herbicide.

In one embodiment, seeds of the present disclosure comprise the herbicide-tolerance characteristics of the herbicide-tolerant plant. In other embodiments, a seed is capable of germination into a plant comprising in at least some of its cells a polynucleotide operably linked to a promoter functional in a plant cell, the promoter capable of expressing a CYP72A219 polypeptide encoded by the polynucleotide, the expression of the CYP72A219 polypeptide conferring to the progeny or descendant plant tolerance to the herbicides.

In some embodiments, plant cells of the present disclosure are capable of regenerating a plant or plant part. In other embodiments, plant cells are not capable of regenerating a plant or plant part. Examples of cells not capable of regenerating a plant include, but are not limited to, endosperm, seed coat (testa and pericarp), and root cap.

In another embodiment, the disclosure refers to a plant cell transformed by a nucleic acid encoding a CYP72A219 polypeptide as described herein, wherein expression of the nucleic acid in the plant cell results in increased resistance or tolerance to an herbicide as compared to a wild-type variety of the plant cell.

Several embodiments provide a commodity plant product prepared from the herbicide-tolerant plants. Examples of commodity plant products include, without limitation, grain, oil, and meal. In one embodiment, a plant product is plant grain (e.g., grain suitable for use as feed or for processing), plant oil (e.g., oil suitable for use as food or biodiesel), or plant meal (e.g., meal suitable for use as feed). For example, the commodity plant product can comprise fodder, seed meal, oil, milk (e.g., soy milk), flour (e.g., soy flour), grits, protein (e.g., protein concentrate, hydrolyzed vegetable protein, textured vegetable protein), tofu, miso, tempeh, fiber, starch, bio-composite building materials (e.g., particleboard, laminated plywood, or lumber products), or seed-treatment-coated seeds. The commodity plant products can comprise the CYP72A219 nucleic acid or CYP72A219 protein.

A commodity plant product prepared from a plant or plant part is provided, wherein the plant or plant part comprises in at least some of its cells a polynucleotide operably linked to a promoter functional in plant cells, the promoter capable of expressing a CYP72A219 polypeptide encoded by the polynucleotide, the expression of the CYP72A219 polypeptide conferring to the plant or plant part tolerance to the herbicide.

The commodity plant product may be produced at the site where the plant has been grown, the plants and/or parts thereof may be removed from the site where the plants have been grown to produce the product. Typically, the plant is grown, the desired harvestable parts are removed from the plant, if feasible in repeated cycles, and the product made from the harvestable parts of the plant. The step of growing the plant may be performed only once each time the method is performed, while allowing repeated times the steps of product production e.g., by repeated removal of harvestable parts of the plants of the disclosure and if necessary further processing of these parts to arrive at the product. It is also possible that the step of growing the plants is repeated and plants or harvestable parts are stored until the production of the product is then performed once for the accumulated plants or plant parts. Also, the steps of growing the plants and producing the product may be performed with an overlap in time, even simultaneously to a large extent or sequentially. Generally, the plants are grown for some time before the product is produced.

### Gene Stacking

The CYP72A219 genes of the disclosure can be stacked with any combination of polynucleotide sequences of interest in order to create plants with a desired phenotype. For example, the CYP72A219 genes may be stacked with one or more additional herbicide tolerance genes, including one or more additional HPPD inhibitor herbicide tolerance genes.

By way of example, polynucleotides that may be stacked with the CYP72A219 genes include nucleic acids encoding polypeptides conferring resistance to pests/pathogens such as viruses, nematodes, insects or fungi, and the like. Illustrative polynucleotides that may be stacked with the CYP72A219 genes of the disclosure include polynucleotides encoding: polypeptides having pesticidal and/or insecticidal activity, such as Bacillus thuringiensis toxic proteins (described in U.S. Pat. Nos. 5,366,892; 5,747,450; 5,737,514; 5,723,756; 5,593,881; and Geiser et al., (1986) Gene 48:109), lectins (Van Damme et al. (1994) Plant Mol. Biol. 24:825, pentin (described in U.S. Pat. No. 5,981,722), and the like; traits desirable for disease or herbicide resistance (e.g., fumonisin detoxification genes (U.S. Pat. No. 5,792,931); avirulence and disease resistance genes (Jones et al. (1994) Science 266:789; Martin et al., (1993) Science 262:1432; Mindrinos et al. (1994) Cell 78:1089); acetolactate synthase (ALS) mutants that lead to herbicide resistance such as the S4 and/or Hra mutations; glyphosate resistance (e.g., 5-enol-pyrovyl-shikimate-3-phosphate-synthase (EPSPS) gene, described in U.S. Pat. Nos. 4,940,935 and 5,188,642; or the glyphosate N-acetyltransferase (GAT) gene, described in Castle et al. (2004) Science, 304:1151-1154; and in U.S. Patent App. Pub. Nos. 20070004912, 20050246798, and 20050060767)); glufosinate resistance (e.g, phosphinothricin acetyl transferase genes PAT and BAR, described in U.S. Pat. Nos. 5,561,236 and 5,276,268); resistance to herbicides including sulfonyl urea, DHT (2,4D), and PPO herbicides (e.g., glyphosate acetyl transferase, aryloxy alkanoate dioxygenase, acetolactate synthase, and protoporphyrinogen oxidase); other cytochrome P450s that confer herbicide tolerance (U.S. patent application Ser. No. 12/156,247; U.S. Pat. Nos. 6,380,465; 6,121,512; 5,349,127; 6,649,814; and 6,300,544; and PCT Patent App. Pub. No. WO2007000077); and traits desirable for processing or process products such as high oil (e.g., U.S. Pat. No. 6,232,529); modified oils (e.g., fatty acid desaturase genes (U.S. Pat. No. 5,952,544; WO 94/11516)); and modified starches (e.g., ADPG pyrophosphorylases (AGPase), starch synthases (SS), starch branching enzymes (SBE), and starch debranching enzymes (SDBE)); the disclosures of which are herein incorporated by reference.

In certain embodiments, the plant comprises at least one additional herbicide-tolerant trait selected, for example, from 5-enolpyruvylshikimate-3-phosphate synthase (EPSPS), Glyphosate acetyl transferase (GAT), phosphinothricin acetyltransferase (PAT), Acetohydroxyacid synthase (AHAS; EC 4.1.3.18, also known as acetolactate synthase or ALS), hydroxyphenyl pyruvate dioxygenase (HPPD), Phytoene desaturase (PD) and dicamba degrading enzymes as disclosed in WO 02/068607, or phenoxyacetic acid- and phenoxypropionic acid-derivative degrading enzymes as disclosed in WO 2008141154 or WO 2005107437.

### Use in Breeding Methods

The plants of the disclosure may be used in a plant breeding program. The goal of plant breeding is to combine, in a single variety or hybrid, various desirable traits. For field crops, these traits may include, for example, resistance to diseases and insects, tolerance to heat and drought, tolerance to chilling or freezing, reduced time to crop maturity, greater yield and better agronomic quality. With mechanical harvesting of many crops, uniformity of plant characteristics such as germination and stand establishment, growth rate, maturity and plant and ear height is desirable. Traditional plant breeding is an important tool in developing new and improved commercial crops. This disclosure encompasses methods for producing a plant by crossing a first parent plant with a second parent plant wherein one or both of the parent plants is a plant displaying a phenotype as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids and transformation. Often combinations of these techniques are used.

The development of hybrids in a plant breeding program requires, in general, the development of homozygous inbred lines, the crossing of these lines and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed plant to an elite inbred line and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly homozygous and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained.

Plants of the present disclosure may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B) times (C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted.

### Herbicide Resistant Weed Control

Several embodiments provide compositions and methods for controlling the growth of an herbicide resistant weed at a plant cultivation site by contacting the weed with a composition that reduces expression or activity of a CYP72A219 polypeptide.

In certain embodiments, the compositions comprise a polynucleotide that reduces expression or activity of the CYP72A219 polypeptide. Systemic regulation (e.g., systemic suppression or silencing) of a target CYP72A219 gene in a plant can be by topical application to the plant of a polynucleotide molecule with a segment in a nucleotide sequence essentially identical to, or essentially complementary to, a sequence of 18 or more contiguous nucleotides in either the target CYP72A219 gene or RNA transcribed from the target CYP72A219 gene, whereby the composition permeates the interior of the plant and induces systemic regulation of the target CYP72A219 gene by the action of single-stranded RNA that hybridizes to the transcribed RNA, e.g., messenger RNA.

The polynucleotides are designed to induce systemic regulation or suppression of an endogenous gene in a plant and are designed to have a sequence essentially identical or essentially complementary to the sequence (which can be coding sequence or non-coding sequence) of an endogenous CYP72A219 gene of a resistant plant or to the sequence of RNA transcribed from an endogenous CYP72A219 gene of a resistant plant. By “essentially identical” or “essentially complementary” is meant that the polynucleotides (or at least one strand of a double-stranded polynucleotide) are designed to hybridize under physiological conditions in cells of the plant to the endogenous gene or to RNA transcribed from the endogenous gene to effect regulation or suppression of the endogenous gene.

In certain embodiments, the compositions and methods can comprise permeability-enhancing agents and treatments to condition the surface of plant tissue, e.g., leaves, stems, roots, flowers, or fruits, to permeation by the polynucleotides into plant cells. The transfer of polynucleotides into plant cells can be facilitated by the prior or contemporaneous application of a polynucleotide to the plant tissue. In some embodiments the permeability-enhancing agent is applied subsequent to the application of the polynucleotide composition. The permeability-enhancing agent enables a pathway for polynucleotides through cuticle wax barriers, stomata and/or cell wall or membrane barriers and into plant cells. Suitable agents to facilitate transfer of the composition into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof.

Chemical agents for conditioning include (a) surfactants, (b) an organic solvent or an aqueous solution or aqueous mixtures of organic solvents, (c) oxidizing agents, (e) acids, (f) bases, (g) oils, (h) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Such agents for conditioning of a plant to permeation are applied to the plant by any convenient method, e.g., spraying or coating with a powder, emulsion, suspension, or solution; similarly, the polynucleotide molecules are applied to the plant by any convenient method, e.g., spraying or wiping a solution, emulsion, or suspension.

### Detection Tools

Several embodiments provide a method for identifying an herbicide-resistant plant, or cells or tissues thereof. In some embodiments, the method includes using primers or probes which specifically recognize a portion of the sequence of the gene.

In an embodiment, the method is based on identifying the expression level of a CYP72A219 gene in the plant. In some embodiments, a PCR-based technique is used to quantify the expression of a CYP72A219 gene that is differentially expressed in resistant plants compared to sensitive plants prior to treatment. In other words, basal expression levels are heightened in resistant plants compared to sensitive plants prior to herbicide treatment. In another embodiment, the method is based on detecting one or more motifs present in the promoter of the CYP72A219 gene in the plant. In certain embodiments, the motif comprises the nucleotide sequence of SEQ ID NO: 34, 35, 36, 37, 38, 39, 40, 41, 42, 43, or 44, or 45. Plants can be identified as herbicide-resistant based on the presence of SEQ ID NO: 34, 35, 36, 37, 38, 39, 40, or 41 or the absence of SEQ ID NO: 42, 43, or 44 in the promoter of the CYP72A219 gene.

In some embodiments, the identification is performed using polymerase chain reaction. The method may also include providing a detectable marker specific to the CYP72A219 gene. In embodiments, the detection is performed using an Enzyme-Linked Immunosorbent Assay (ELISA), a quantitative real-time polymerase chain reaction (qPCR), or an RNA-hybridization technique.

In some embodiments, the kits include a specific probe having a sequence which corresponds to or is complementary to a sequence having between 80% and 100% sequence identity with a specific region of the CYP72A219 gene. In some embodiments, the kit includes a specific probe which corresponds to or is complementary to a sequence having between 90% and 100% sequence identity with a specific region of the CYP72A219 gene.

The methods, kits, and primers can be used for different purposes including, but not limited to the following: identifying the presence or absence of herbicide resistance in plants, plant material such as seeds or cuttings; determining the presence of herbicide-resistant weeds in crop fields; and tailoring an herbicide regime to effectively and economically manage weeds affecting agricultural crops.

### Embodiments

The following numbered embodiments also form part of the present disclosure:

1. A modified plant, or a progeny, plant seed, plant part, or plant cell thereof, having tolerance to an herbicide, the modified plant having increased expression of a polynucleotide encoding a cytochrome P450 72A219 (CYP72A219) polypeptide relative to an unmodified plant, progeny, plant seed, plant part, or plant cell of the same species.

2. The modified plant, progeny, plant seed, plant part, or plant cell of embodiment 1, wherein the modified plant comprises a heterologous polynucleotide encoding the CYP72A219 polypeptide.

3. The modified plant, progeny, plant seed, plant part, or plant cell of embodiment 1 or embodiment 2, wherein the CYP72A219 polypeptide has at least 80%, at least 90%, at least 95%, at least 98%, at least 99%, or 100% sequence identity to SEQ ID NO: 2 or 4.

4. The modified plant, progeny, plant seed, plant part, or plant cell of any one of embodiments 1-3, wherein the polynucleotide encoding the CYP72A219 polypeptide has at least 80%, at least 90%, at least 95%, at least 98%, at least 99%, or 100% sequence identity to SEQ ID NO: 1 or 3.

5. The modified plant, progeny, plant seed, plant part, or plant cell of any one of embodiments 1-4, wherein the polynucleotide is operably linked to a heterologous promoter functional in a plant cell.

6. The modified plant, progeny, plant seed, plant part, or plant cell of any one of embodiments 1-5, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide, optionally wherein the herbicide is an HPPD inhibitor herbicide.

7. The modified plant, progeny, plant seed, plant part, or plant cell of any one of embodiments 1-6, wherein the HPPD inhibitor herbicide is tembotrione or mesotrione.

8. The modified plant, progeny, plant seed, plant part, or plant cell of any one of embodiments 1-7, wherein the plant is a dicotyledonous or monocotyledonous plant.

9. The modified plant, progeny, plant seed, plant part, or plant cell of any one of embodiments 1-8, wherein the plant is a crop plant.

10. The modified plant, progeny, plant seed, plant part, or plant cell of any one of embodiments 1-9, wherein the plant is a maize, sorghum, wheat, sunflower, rice, soybean, cotton, canola, tobacco, tomato, potato, pepper, barley, alfalfa, sugar cane, or sugar beet plant.

11. The modified plant, progeny, plant seed, plant part, or plant cell of any one of embodiments 1-10, wherein the modified plant further comprises a second herbicide-tolerant trait.

12. The modified plant, progeny, plant seed, plant part, or plant cell of any one of embodiments 1-11, wherein the plant, progeny, plant seed, plant part, or plant cell is non-viable and/or non-regenerable.

13. A nucleic acid molecule comprising: (a) a nucleotide sequence encoding a CYP72A219 polypeptide, wherein the nucleotide sequence has at least 80%, at least 90%, at least 95%, at least 98%, at least 99%, or 100% sequence identity to SEQ ID NO: 1 or 3; or (b) a nucleotide sequence encoding a CYP72A219 polypeptide, wherein the CYP72A219 polypeptide has at least 80%, at least 90%, at least 95%, at least 98%, at least 99%, or 100% sequence identity to SEQ ID NO: 2 or 4.

14. The nucleic acid molecule of embodiment 13, wherein the nucleic acid molecule is an isolated, synthetic, or recombinant nucleic acid molecule.

15. An expression cassette comprising the nucleic acid molecule of embodiment 13 or embodiment 14 operably linked to a heterologous promoter functional in a plant cell.

16. A vector comprising the nucleic acid molecule of embodiment 13 or embodiment 14, or the expression cassette of embodiment 15.

17. A CYP72A219 polypeptide comprising an amino acid sequence having at least 80%, at least 90%, at least 95%, at least 98%, at least 99%, or 100% sequence identity to SEQ ID NO: 2 or 4.

18. A plant, plant part, plant seed, or plant cell comprising the nucleic acid molecule of embodiment 13 or embodiment 14, the expression cassette of embodiment 15, the vector of embodiment 16, or the polypeptide of embodiment 17.

19. A biological sample comprising the nucleic acid molecule of embodiment 13 or embodiment 14, the expression cassette of embodiment 15, the vector of embodiment 16, or the polypeptide of embodiment 17.

20. A method for producing a plant with herbicide tolerance, the method comprising: increasing expression of a polynucleotide encoding a CYP72A219 polypeptide in the plant, wherein the herbicide tolerance of the plant is increased when compared to a plant that lacks the increased expression.

21. The method of embodiment 20 comprising introducing to a plant cell a polynucleotide encoding the CYP72A219 polypeptide, wherein the polynucleotide is operably linked to a heterologous promoter functional in a plant cell; and regenerating a plant from the plant cell.

22. The method of embodiment 20 or embodiment 21, wherein the CYP72A219 polypeptide has at least 80%, at least 90%, at least 95%, at least 98%, at least 99%, or 100% sequence identity to SEQ ID NO: 2 or 4.

23. The method of any one of embodiments 20-22, wherein the polynucleotide encoding the CYP72A219 polypeptide has at least 80%, at least 90%, at least 95%, at least 98%, at least 99%, or 100% sequence identity to SEQ ID NO: 1 or 3.

24. The method of any one of embodiments 20-23, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide, optionally wherein the herbicide is an HPPD inhibitor herbicide.

25. The method of any one of embodiments 20-24, wherein the HPPD inhibitor herbicide is tembotrione or mesotrione.

26. The method of any one of embodiments 20-25, wherein the plant is a dicotyledonous or monocotyledonous plant.

27. The method of any one of embodiments 20-26, wherein the plant is a crop plant.

28. The method of any one of embodiments 20-27, wherein the plant is a maize, sorghum, wheat, sunflower, rice, soybean, cotton, canola, tobacco, tomato, potato, pepper, barley, alfalfa, sugar cane, or sugar beet plant.

29. A method for controlling undesired vegetation at a plant cultivation site, the method comprising: providing at the site a plant that comprises a polynucleotide encoding a CYP72A219 polypeptide, wherein expression of the polynucleotide confers to the plant tolerance to an herbicide; and applying to the site an effective amount of the herbicide.

30. The method of embodiment 29, wherein the CYP72A219 polypeptide has at least 80%, at least 90%, at least 95%, at least 98%, at least 99%, or 100% sequence identity to SEQ ID NO: 2 or 4.

31. The method of embodiment 29 or embodiment 30, wherein the polynucleotide encoding the CYP72A219 polypeptide has at least 80%, at least 90%, at least 95%, at least 98%, at least 99%, or 100% sequence identity to SEQ ID NO: 1 or 3.

32. The method of any one of embodiments 29-31, wherein the polynucleotide is operably linked to a heterologous promoter functional in a plant cell.

33. The method of any one of embodiments 29-32, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide, optionally wherein the herbicide is an HPPD inhibitor herbicide.

34. The method of any one of embodiments 29-33, wherein the HPPD inhibitor herbicide is tembotrione or mesotrione.

35. The method of any one of embodiments 29-34, wherein the plant is a dicotyledonous or monocotyledonous plant.

36. The method of any one of embodiments 29-35, wherein the plant is a maize, sorghum, wheat, sunflower, rice, soybean, cotton, canola, tobacco, tomato, potato, pepper, barley, alfalfa, sugar cane, or sugar beet plant.

37. A method for controlling the growth of an herbicide resistant weed at a plant cultivation site, the method comprising: contacting the weed with a composition that reduces expression or activity of a CYP72A219 polypeptide; and applying to the site an effective amount of the herbicide.

38. The method of embodiment 37, wherein the composition comprises a polynucleotide that reduces expression or activity of a CYP72A219 polypeptide.

39. The method of embodiment 38, wherein the polynucleotide is a double-stranded RNA, a single-stranded RNA, or a double-stranded DNA/RNA hybrid polynucleotide.

40. The method of embodiment 38 or embodiment 39, wherein the polynucleotide comprises a sequence essentially identical or essentially complementary to at least 18 or more contiguous nucleotides of SEQ ID NO: 1 or 3.

41. The method of any one of embodiments 38-40, wherein the polynucleotide has a length of 26-60 nucleotides.

42. The method of any one of embodiments 37-41, wherein the CYP72A219 polypeptide has at least 80%, at least 90%, at least 95%, at least 98%, at least 99%, or 100% sequence identity to SEQ ID NO: 2 or 4.

43. The method of any one of embodiments 37-42, wherein the composition comprises a chemical inhibitor that reduces expression or activity of the CYP72A219 polypeptide.

44. The method of any one of embodiments 37-43, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide, optionally wherein the herbicide is an HPPD inhibitor herbicide.

45. The method of any one of embodiments 37-44, wherein the HPPD inhibitor herbicide is tembotrione or mesotrione.

46. The method of any one of embodiments 37-45, wherein the weed is Amaranthus palmeri.

47. The method of any one of embodiments 37-46, wherein the composition comprises a permeability-enhancing agent.

48. A commodity plant product prepared from the plant, plant part, plant seed, or plant cell of any one of embodiments 1-12, wherein the product comprises the CYP72A219 polypeptide or the polynucleotide encoding the CYP72A219 polypeptide.

49. The commodity plant product of embodiment 48, wherein the product comprises fodder, seed meal, oil, milk, flour, grits, protein, tofu, miso, tempeh, fiber, starch, bio-composite building materials or seed-treatment-coated seed.

50. A method for producing a commodity plant product, the method comprising processing the plant or plant part of any one of embodiments 1-12 to obtain the product.

51. The method of embodiment 50, wherein the product comprises the CYP72A219 polypeptide or the polynucleotide encoding the CYP72A219 polypeptide.

52. The method of embodiment 50 or embodiment 51, wherein the plant product comprises fodder, seed meal, oil, milk, flour, grits, protein, tofu, miso, tempeh, fiber, starch, bio-composite building materials or seed-treatment-coated seeds.

53. A method for identifying an herbicide-resistant plant, the method comprising: obtaining a sample from a plant suspected of having herbicide resistance; quantifying expression of a CYP72A219 gene in the sample, wherein the CYP72A219 gene is differentially expressed in an herbicide-resistant plant compared to an herbicide-sensitive plant of the same species; and determining that the plant is herbicide-resistant based on the quantification.

54. The method of embodiment 53, wherein the sample is from Amaranthus palmeri.

55. The method of embodiment 53, wherein the sample is from a maize, sorghum, wheat, sunflower, rice, soybean, cotton, canola, tobacco, tomato, potato, pepper, barley, alfalfa, sugar cane, or sugar beet plant.

56. The method of any one of embodiments 53-55, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide, optionally wherein the herbicide is an HPPD inhibitor herbicide.

57. The method of any one of embodiments 53-56, wherein the quantifying expression of the CYP72A219 gene comprises quantifying CYP72A219 mRNA.

58. The method of any one of embodiments 53-57, wherein the quantifying expression of the CYP72A219 gene comprises quantifying CYP72A219 polypeptide.

59. The method of any one of embodiments 53-58, wherein the sample is a nucleic acid or protein sample.

60. The method of any one of embodiments 53-59, wherein the CYP72A219 gene has at least 80%, at least 90%, at least 95%, at least 98%, at least 99%, or 100% sequence identity to SEQ ID NO: 1 or 3.

61. The method of any one of embodiments 53-60, wherein the quantifying expression comprises amplifying a nucleic acid using at least two primers.

62. A method for identifying an herbicide-resistant plant, the method comprising: providing a sample from a plant suspected of having herbicide resistance; detecting one or more motifs in the promoter of a CYP72A219 gene in the sample, wherein the motif comprises the nucleotide sequence of SEQ ID NO: 34, 35, 36, 37, 38, 39, 40, or 41; and determining that the plant is herbicide-resistant based on the presence of the one or more motifs.

63. The method of embodiment 62, wherein the CYP72A219 gene has at least 80%, at least 90%, at least 95%, at least 98%, at least 99%, or 100% sequence identity to SEQ ID NO: 1 or 3.

64. The method of embodiment 62 or embodiment 63, wherein the detecting comprises amplifying a nucleic acid using at least two primers.

65. A kit for identifying an herbicide-resistant plant, the kit comprising at least two primers, wherein the at least two primers recognize a CYP72A219 gene that is differentially expressed in an herbicide-resistant plant compared to an herbicide-sensitive plant of the same species.

66. The kit of embodiment 65, wherein the CYP72A219 gene has at least 80%, at least 90%, at least 95%, at least 98%, at least 99%, or 100% sequence identity to SEQ ID NO: 1 or 3.

67. The kit of embodiment 65 or embodiment 66, wherein the primers recognize one or more motifs in the promoter of the CYP72A219 gene.

68. The kit of any one of embodiments 65-67, further comprising at least one of a positive control and a negative control.

69. The kit of any one of embodiments 65-68, further comprising components of a qRT-PCR solution.

70. The kit of any one of embodiments 65-69, wherein the plant is Amaranthus palmeri.

71. The kit of any one of embodiments 65-69, wherein the plant is a maize, sorghum, wheat, sunflower, rice, soybean, cotton, canola, tobacco, tomato, potato, pepper, barley, alfalfa, sugar cane, or sugar beet plant.

72. The kit of any one of embodiments 65-71, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide, optionally wherein the herbicide is an HPPD inhibitor herbicide.

All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, it will be obvious that certain changes and modifications may be practiced within the scope of the appended claims.

The following examples are offered by way of illustration and not by way of limitation.

### EXAMPLES

### Example 1: Identification of Cytochrome P450 Genes Involved in Metabolism of Tembotrione in Palmer Amaranth

This example aimed to identify important cis-regulatory specific sequences in the promoter of cytochrome P450 genes in a Palmer amaranth (Amaranthus palmeri) population from Nebraska resistant to herbicides that inhibit 4-hydroxyphenylpyruvate dioxygenase, including tembotrione and mesotrione. This HPPD-resistant population is referred to as NER. The population NER was previously shown to rapidly detoxify the herbicide tembotrione to a 4-hydroxy tembotrione metabolite followed by glycosylation. This hydroxylation occurred more rapidly in NER than in an HPPD-susceptible population from the same field site in Nebraska referred to as NES.

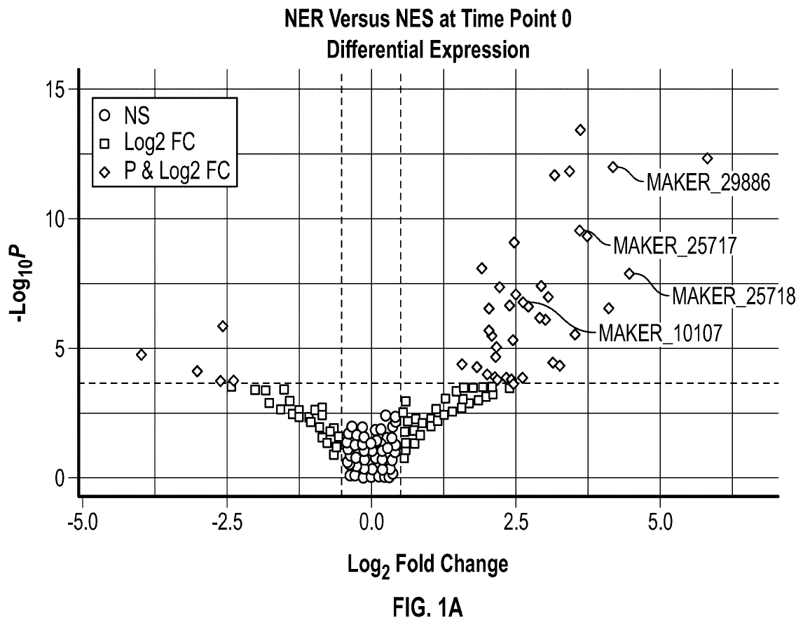

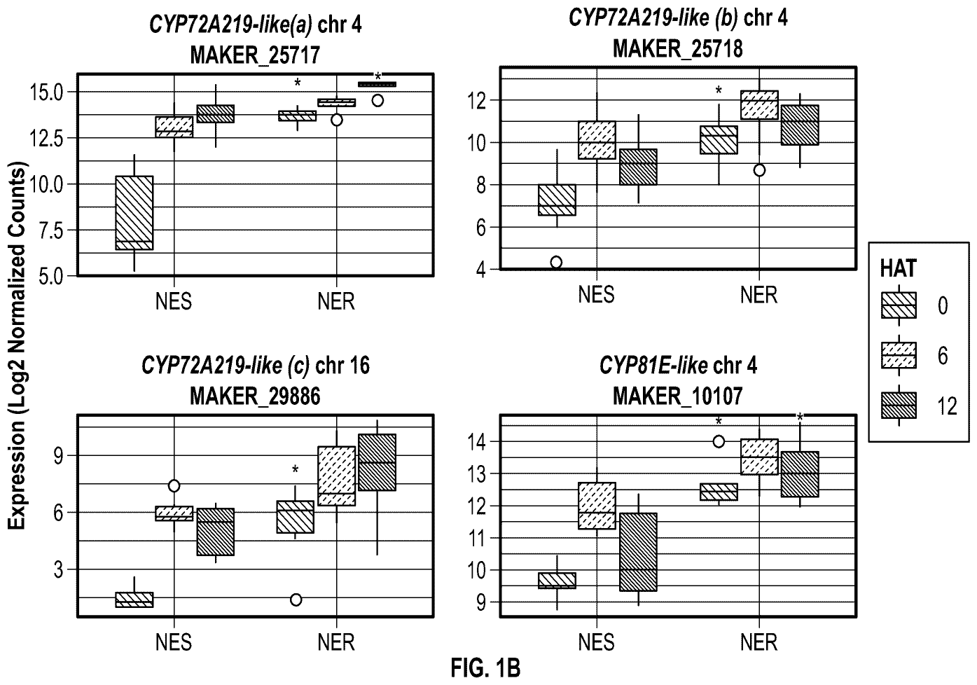

An RNAseq experiment was used to analyze differential gene expression between NER and NES. The gene expression analysis identified the genes CYP81E and CYP72A219 as candidate genes for HPPD herbicide metabolism having higher constitutive expression in NER than in NES (FIG. 1A). Three different genes annotated as CYP72A219 (MAKER_25717, MAKER_25718, and MAKER_29886) were identified in the Palmer amaranth reference genome, along with one gene annotated as CYP81E (MAKER_10107). Expression of all four genes was induced in NES following tembotrione treatment, consistent with the formation of similar 4-hydroxy tembotrione metabolites in NES but at a slower rate than in NER (FIG. 1B). qRT-PCR confirmed the high expression of both genes in parental lines at 3 and 6 h after treatment with tembotrione (data not shown). No sequence variants in these genes from RNAseq were associated with resistance, and this result was confirmed by Sanger sequencing.

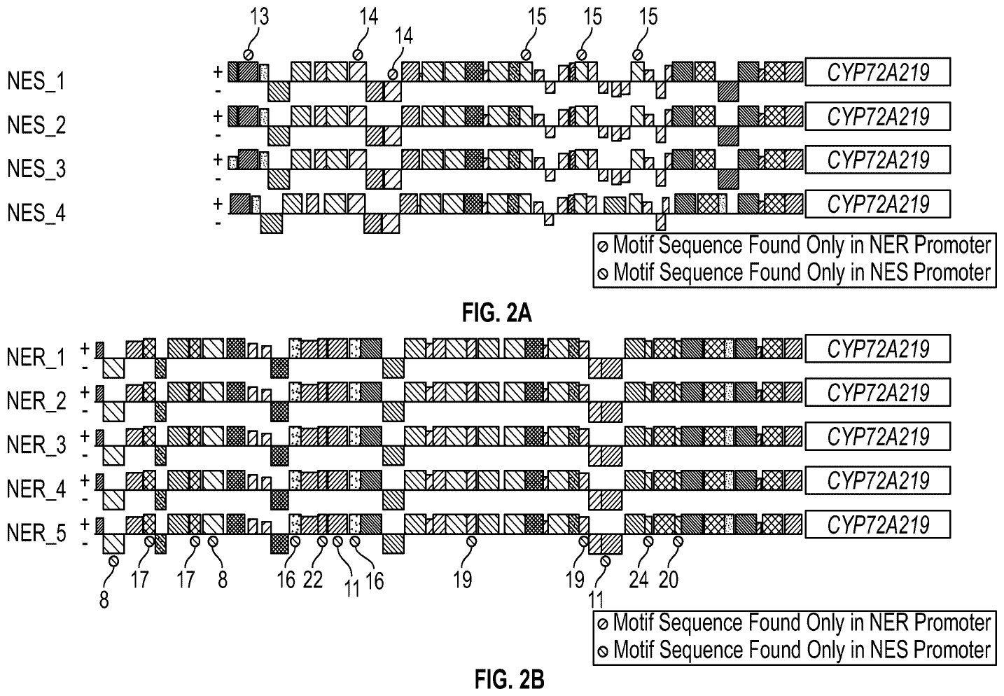

Primers to amplify the promoters of both genes were designed after alignment with the available genome of Palmer amaranth. The samples used to sequence the promoter were five resistant (R) and four susceptible(S) plants from an F2 population produced by crossing NER and NES. The sequenced promoters were subjected to analysis for both common and unique motifs using the Multiple Expectation maximizations for Motif Elicitation (MEME-suite) tool. Gene Ontology for Motifs (GOMo) tool was used to determine if any motif was significantly associated with genes linked to one or more genome ontology (Go). The DNA length amplified upstream of CYP81E was 1250 bp for S and R. Primers for the promoters for the first CYP72A219 gene on chromosome four, MAKER-25717, amplified 1380 bp and 1700 bp for S (SEQ ID NOs: 16-19) and R (SEQ ID NOs: 11-15), respectively. Twelve significant binding-site motifs were found in the promoter sequences of CYP81E; however, all were found in both S and R promoter sequences. Twenty-three motifs were found in the CYP72A219 promoter sequences, of which three were specific to the S individuals (SEQ ID NOs: 42-44), eight were specific to the R individuals (SEQ ID NOs: 34-41), and 12 were shared by all S and R individuals (FIG. 2). In the R promoter of CYP72A219 two binding site motifs for MYB transcription factors (TF) were found. The other six motif functions were not determined using GOMo. In the S promoter, a binding site motif of Ethylene Response Factor transcription factor and ATB1 Interacting Factor were identified. Motifs for activators or repressors were not identified, although six motifs in the R promoter had no function determined. The TF binding-sites identified were not related to a stress response. Based on these results, it appears that the high expression of CYP81E in HPPD-resistant Palmer amaranth may be regulated by a trans-regulatory element. The constitutive increased expression of CYP72A219 may be cis-regulated due to differences in the promoter that co-segregate with resistance in the F2.

### Example 2: Validation of Candidate Cytochrome P450s Using a Yeast System

A functional validation of the candidate genes was conducted in a yeast system. In brief, the yeast lines WAT11 and WAT21 express the plant cytochrome P450 reductase genes ATR1 and ATR2, respectively, and have the endogenous yeast cytochrome P450 reductase gene knocked out. The sequences of the three CYP72A219 genes and the CYP81E gene were codon-optimized for expression in yeast and synthesized by Genewiz in vector pUC57. The genes were cloned into yeast expression vector pYES2 with a galactose inducible promoter, a uracil synthesis gene, and an ampicillin resistance gene. The vectors were first transformed into E. coli and selected on ampicillin, followed by sequencing to confirm the correct insertion of the P450 gene sequence. Plasmid was extracted from transformed colonies for transformation into yeast lines WAT11 and WAT21 using a LiAc protocol and plated on glucose-containing media lacking uracil to select for transformed cells. The empty pYES2 vector was included as a negative control, and a vector containing the Nsf1 gene from corn was included as a positive control known to metabolize the HPPD-inhibiting herbicides mesotrione and tembotrione. Transformed yeast lines were incubated with 2% raffinose for 96 h at 30° C. with agitation at 200 rpm until reaching an OD600 of greater than 5. The cells were centrifuged and resuspended with 2 mL of 2% galactose to induce expression of the P450 gene on GAL1 promoter from the pYES2 plasmid. The solutions were incubated with 150 μL of 1000 μM tembotrione for 24 h at 30 C with 200 rpm agitation. The incubation solution was then centrifuged, and the supernatant was analyzed using LC/MS-MS to measure parent tembotrione and hydroxy tembotrione metabolites. A negative control for each plasmid was incubated with 20 mL of 2% galactose at 30 C with 200 rpm agitation.

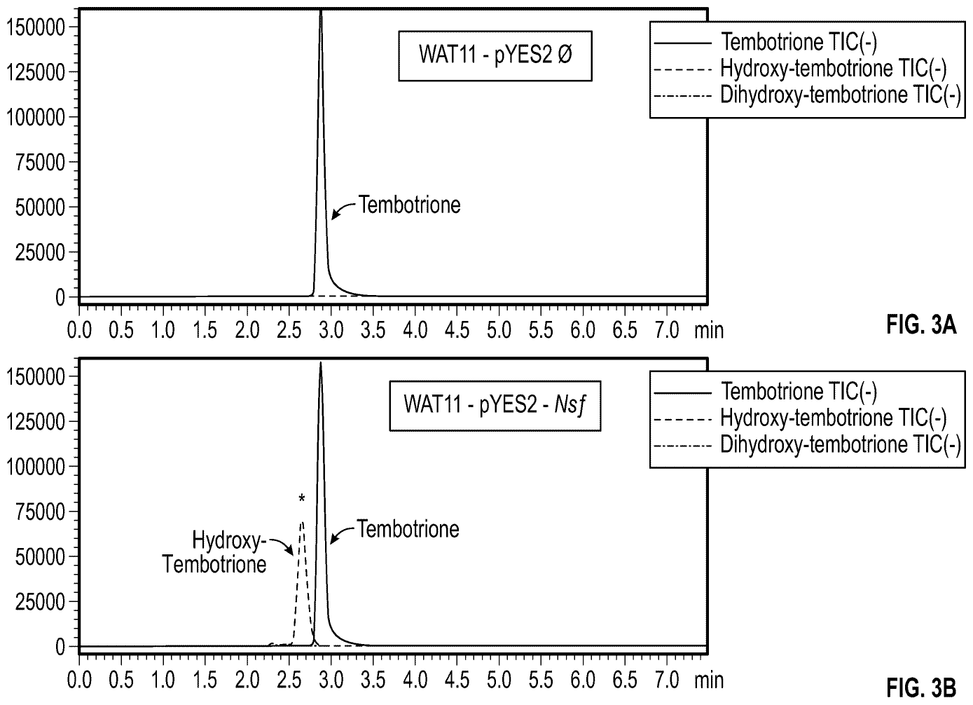

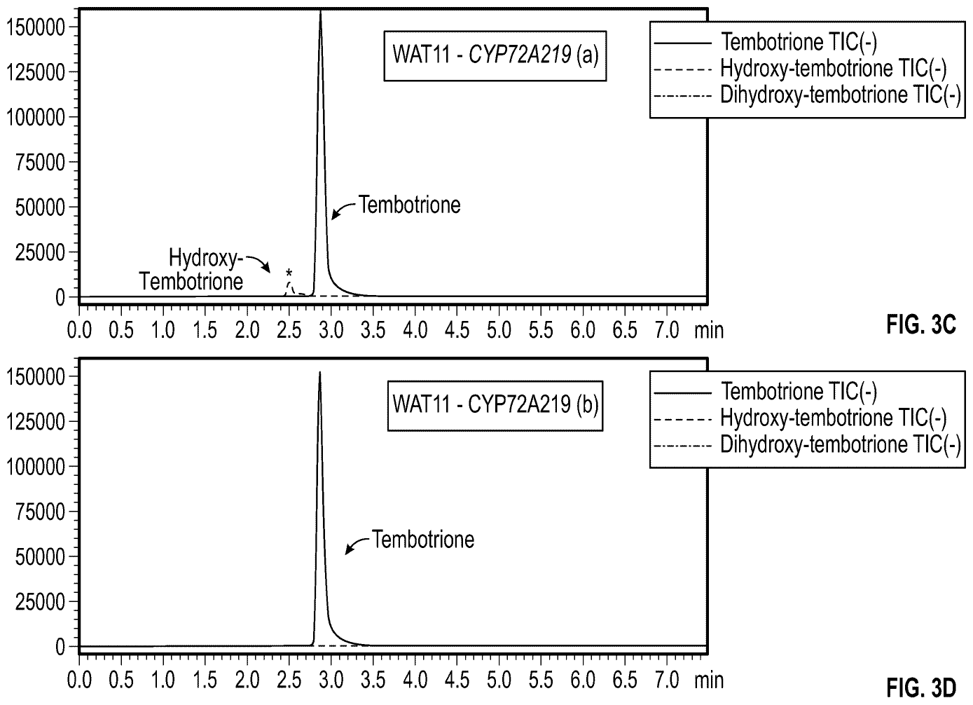

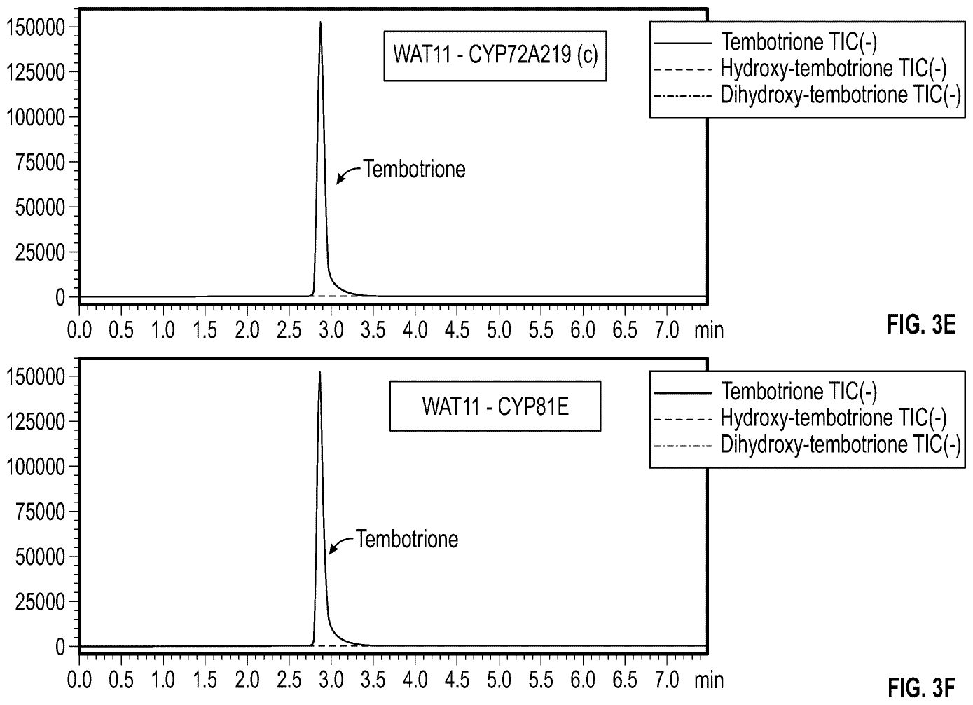

The negative control empty vector showed only intact tembotrione and no metabolites, indicating that the yeast cells did not detoxify tembotrione (FIG. 3A). The positive control vector containing the Nsf1 gene from corn formed the expected hydroxy-tembotrione metabolite and had reduced concentration of tembotrione (FIG. 3B). The vector containing the CYP72A219 gene from chromosome 4 (MAKER-25717 in the reference genome sequence; SEQ ID NO: 1) showed formation of the hydroxy-tembotrione metabolite (FIG. 3C). A variant allele of this CYP72A219 gene (SEQ ID NO: 3) also showed formation of the hydroxy-tembotrione metabolite, providing a biological replication of the results. The second CYP72A219 gene from chromosome 4 (SEQ ID NO: 5) did not show formation of hydroxy-tembotrione, nor did the CYP72A219 gene from chromosome 16 (SEQ ID NO: 7) (FIG. 3D-E). The vector containing the CYP81E gene (SEQ ID NO: 9) also did not show formation of hydroxy-tembotrione (FIG. 3F).

### Example 3: CYP72A219 Expression in HPPD Resistant Palmer Amaranth Populations

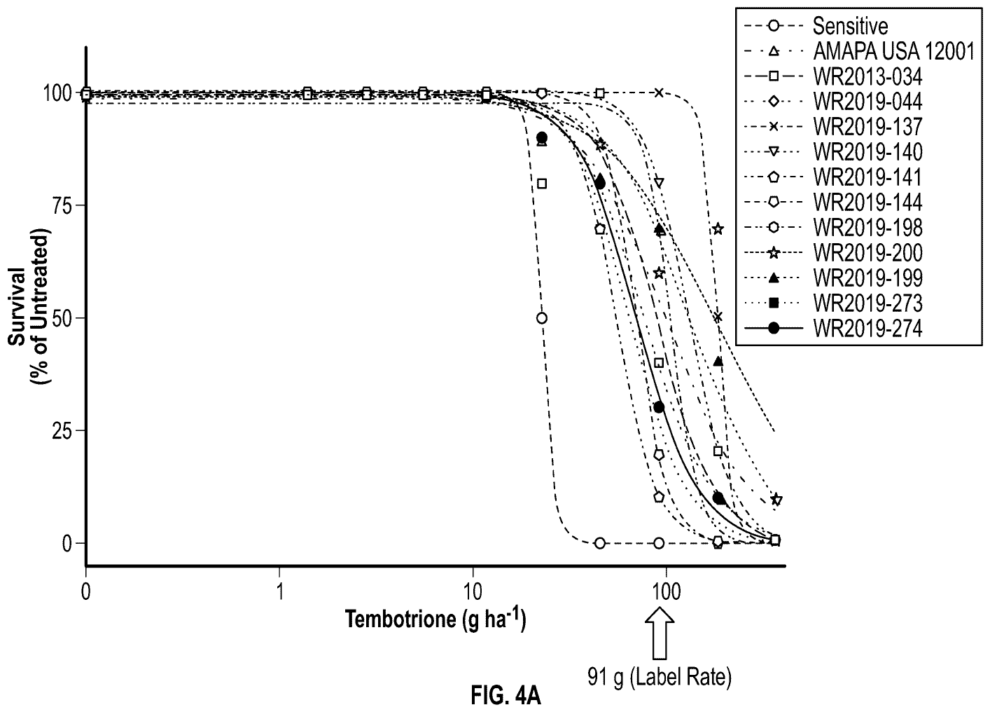

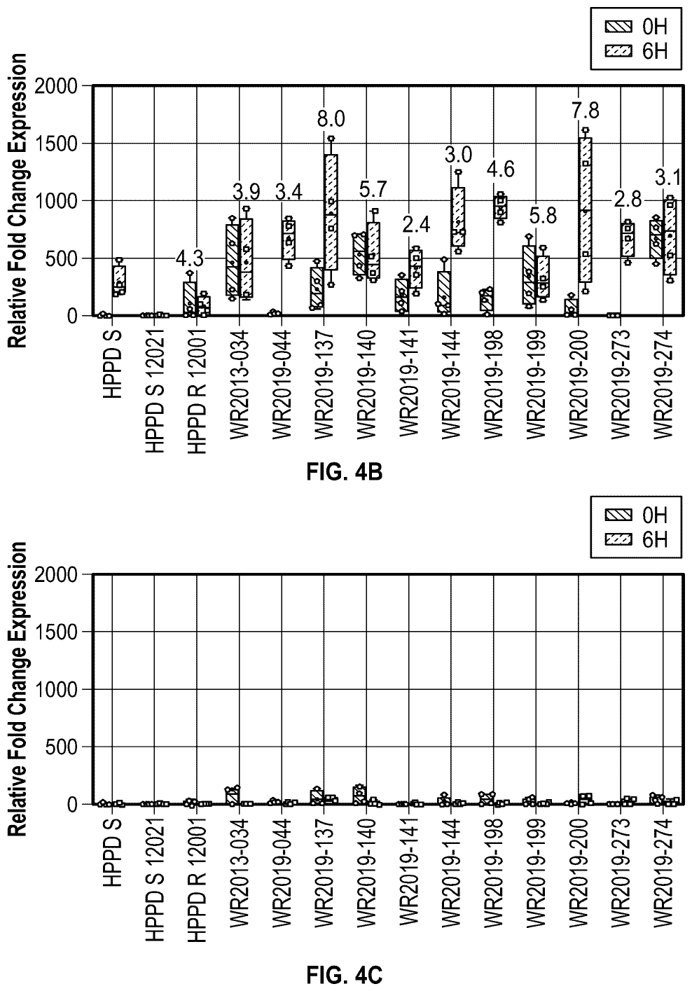

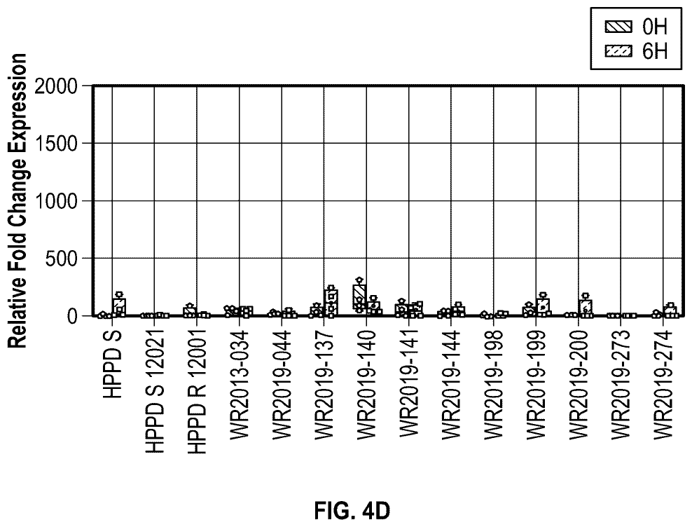

A survey of Palmer amaranth in the US was conducted to identify additional populations resistant to the HPPD inhibiting herbicide tembotrione. A tembotrione dose response was conducted to compare the response of the survey populations to a known sensitive population. The population USA 12001 (the population used for all previous work to identify CYP450 genes) was used as a known resistant reference. Eleven populations from the survey were identified that had a significantly higher LD50 (dose of tembotrione required to cause 50% mortality in the population) than a reference sensitive population (FIG. 4A).

Transcript abundance was measured in the 11 resistant survey populations for genes CYP72A219 (a), CYP81E, and a CYP not implicated in herbicide resistance (FIG. 4B-D). Known sensitive populations HPPD S and HPPD S 12021 were included as references, as was known resistant population HPPD R 12001, the population previously characterized for increased expression of CYP72A219 (a) and CYP81E. Relative transcript abundance was quantified using qRT-PCR on cDNA synthesized from RNA extracted from individuals before and 6 h after tembotrione treatment, using normalization genes as previously described. Constitutively increased expression of CYP72A219 (a) in the HPPD R 12001 population relative to the known sensitive populations was confirmed. All 11 resistant populations from the survey had increased CYP72A219 (a) expression following tembotrione treatment, and most had constitutively increased expression. No resistant populations from the survey had increased expression of CYP81E, either constitutively or after tembotrione treatment. The CYP unrelated to herbicide resistance also had not increased expression in any populations, whether before or after tembotrione treatment. These results show that the gene CYP72A219 (a) is consistently upregulated in tembotrione resistant Palmer amaranth, in populations that are genetically unrelated.

What is claimed is:

1. A modified plant, or a progeny, plant seed, plant part, or plant cell thereof, having tolerance to an herbicide, the modified plant having increased expression of a polynucleotide encoding a cytochrome P450 72A219 (CYP72A219) polypeptide relative to an unmodified plant, progeny, plant seed, plant part, or plant cell of the same species.

2. The modified plant, progeny, plant seed, plant part, or plant cell of claim 1, wherein the modified plant comprises a heterologous polynucleotide encoding the CYP72A219 polypeptide.

3. The modified plant, progeny, plant seed, plant part, or plant cell of claim 1, wherein the CYP72A219 polypeptide has at least 80%, at least 90%, at least 95%, at least 98%, at least 99%, or 100% sequence identity to SEQ ID NO: 2 or 4.

4. The modified plant, progeny, plant seed, plant part, or plant cell of claim 1, wherein the polynucleotide encoding the CYP72A219 polypeptide has at least 80%, at least 90%, at least 95%, at least 98%, at least 99%, or 100% sequence identity to SEQ ID NO: 1 or 3.

5. The modified plant, progeny, plant seed, plant part, or plant cell of claim 1, wherein the polynucleotide is operably linked to a heterologous promoter functional in a plant cell.

6. The modified plant, progeny, plant seed, plant part, or plant cell of claim 1, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide.

7. The modified plant, progeny, plant seed, plant part, or plant cell of claim 6, wherein the HPPD inhibitor herbicide is tembotrione or mesotrione.

8. The modified plant, progeny, plant seed, plant part, or plant cell of claim 1, wherein the plant is a dicotyledonous or monocotyledonous plant.

9. The modified plant, progeny, plant seed, plant part, or plant cell of claim 1, wherein the plant is a crop plant.

10. The modified plant, progeny, plant seed, plant part, or plant cell of claim 1, wherein the plant is a maize, sorghum, wheat, sunflower, rice, soybean, cotton, canola, tobacco, tomato, potato, pepper, barley, alfalfa, sugar cane, or sugar beet plant.

11. The modified plant, progeny, plant seed, plant part, or plant cell of claim 1, wherein the modified plant further comprises a second herbicide-tolerant trait.

12. The modified plant, progeny, plant seed, plant part, or plant cell of claim 1, wherein the plant, progeny, plant seed, plant part, or plant cell is non-viable and/or non-regenerable.

13. A nucleic acid molecule comprising:
(a) a nucleotide sequence encoding a CYP72A219 polypeptide, wherein the nucleotide sequence has at least 80%, at least 90%, at least 95%, at least 98%, at least 99%, or 100% sequence identity to SEQ ID NO: 1 or 3; or
(b) a nucleotide sequence encoding a CYP72A219 polypeptide, wherein the CYP72A219 polypeptide has at least 80%, at least 90%, at least 95%, at least 98%, at least 99%, or 100% sequence identity to SEQ ID NO: 2 or 4.

14. The nucleic acid molecule of claim 13, wherein the nucleic acid molecule is an isolated, synthetic, or recombinant nucleic acid molecule.

15. An expression cassette comprising the nucleic acid molecule of claim 13 operably linked to a heterologous promoter functional in a plant cell.

16. A vector comprising the nucleic acid molecule of claim 13.

17. A biological sample comprising the nucleic acid molecule of claim 13.

18. A plant, plant part, plant seed, or plant cell comprising the nucleic acid molecule of claim 13.

19. A CYP72A219 polypeptide comprising an amino acid sequence having at least 80%, at least 90%, at least 95%, at least 98%, at least 99%, or 100% sequence identity to SEQ ID NO: 2 or 4.

20. A method for producing a plant with herbicide tolerance, the method comprising: increasing expression of a polynucleotide encoding a CYP72A219 polypeptide in the plant, wherein the herbicide tolerance of the plant is increased when compared to a plant that lacks the increased expression.

21. The method of claim 20 comprising introducing to a plant cell a polynucleotide encoding the CYP72A219 polypeptide, wherein the polynucleotide is operably linked to a heterologous promoter functional in a plant cell; and regenerating a plant from the plant cell.

22. The method of claim 20, wherein the CYP72A219 polypeptide has at least 80%, at least 90%, at least 95%, at least 98%, at least 99%, or 100% sequence identity to SEQ ID NO: 2 or 4.

23. The method of claim 20, wherein the polynucleotide encoding the CYP72A219 polypeptide has at least 80%, at least 90%, at least 95%, at least 98%, at least 99%, or 100% sequence identity to SEQ ID NO: 1 or 3.

24. The method of claim 20, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide.

25. The method of claim 24, wherein the HPPD inhibitor herbicide is tembotrione or mesotrione.

26. The method of claim 20, wherein the plant is a dicotyledonous or monocotyledonous plant.

27. The method of claim 20, wherein the plant is a crop plant.

28. The method of claim 20, wherein the plant is a maize, sorghum, wheat, sunflower, rice, soybean, cotton, canola, tobacco, tomato, potato, pepper, barley, alfalfa, sugar cane, or sugar beet plant.

29. A method for controlling undesired vegetation at a plant cultivation site, the method comprising:
providing at the site a plant that comprises a polynucleotide encoding a CYP72A219 polypeptide, wherein expression of the polynucleotide confers to the plant tolerance to an herbicide; and
applying to the site an effective amount of the herbicide.

30. The method of claim 29, wherein the CYP72A219 polypeptide has at least 80%, at least 90%, at least 95%, at least 98%, at least 99%, or 100% sequence identity to SEQ ID NO: 2 or 4.

31. The method of claim 29, wherein the polynucleotide encoding the CYP72A219 polypeptide has at least 80%, at least 90%, at least 95%, at least 98%, at least 99%, or 100% sequence identity to SEQ ID NO: 1 or 3.

32. The method of claim 29, wherein the polynucleotide is operably linked to a heterologous promoter functional in a plant cell.

33. The method of claim 29, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide.

34. The method of claim 33, wherein the HPPD inhibitor herbicide is tembotrione or mesotrione.

35. The method of claim 29, wherein the plant is a dicotyledonous or monocotyledonous plant.

36. The method of claim 29, wherein the plant is a maize, sorghum, wheat, sunflower, rice, soybean, cotton, canola, tobacco, tomato, potato, pepper, barley, alfalfa, sugar cane, or sugar beet plant.

37. A method for controlling the growth of an herbicide resistant weed at a plant cultivation site, the method comprising:
contacting the weed with a composition that reduces expression or activity of a CYP72A219 polypeptide; and
applying to the site an effective amount of the herbicide.

38. The method of claim 37, wherein the composition comprises a polynucleotide that reduces expression or activity of a CYP72A219 polypeptide.

39. The method of claim 38, wherein the polynucleotide is a double-stranded RNA, a single-stranded RNA, or a double-stranded DNA/RNA hybrid polynucleotide.

40. The method of claim 38, wherein the polynucleotide comprises a sequence essentially identical or essentially complementary to at least 18 or more contiguous nucleotides of SEQ ID NO: 1 or 3.

41. The method of claim 38, wherein the polynucleotide has a length of 26-60 nucleotides.

42. The method of claim 37, wherein the CYP72A219 polypeptide has at least 80%, at least 90%, at least 95%, at least 98%, at least 99%, or 100% sequence identity to SEQ ID NO: 2 or 4.

43. The method of claim 37, wherein the composition comprises a chemical inhibitor that reduces expression or activity of the CYP72A219 polypeptide.

44. The method of claim 37, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide.

45. The method of claim 44, wherein the HPPD inhibitor herbicide is tembotrione or mesotrione.

46. The method of claim 37, wherein the weed is Amaranthus palmeri.

47. The method of claim 37, wherein the composition comprises a permeability-enhancing agent.

48. A commodity plant product prepared from the plant, plant part, plant seed, or plant cell of claim 1, wherein the product comprises the CYP72A219 polypeptide or the polynucleotide encoding the CYP72A219 polypeptide.

49. The commodity plant product of claim 48, wherein the product comprises fodder, seed meal, oil, milk, flour, grits, protein, tofu, miso, tempeh, fiber, starch, bio-composite building materials or seed-treatment-coated seed.

50. A method for producing a commodity plant product, the method comprising processing the plant or plant part of claim 1 to obtain the product.

51. The method of claim 50, wherein the product comprises the CYP72A219 polypeptide or the polynucleotide encoding the CYP72A219 polypeptide.

52. The method of claim 50, wherein the plant product comprises fodder, seed meal, oil, milk, flour, grits, protein, tofu, miso, tempeh, fiber, starch, bio-composite building materials, or seed-treatment-coated seeds.

53. A method for identifying an herbicide-resistant plant, the method comprising:
obtaining a sample from a plant suspected of having herbicide resistance;
quantifying expression of a CYP72A219 gene in the sample, wherein the CYP72A219 gene is differentially expressed in an herbicide-resistant plant compared to an herbicide-sensitive plant of the same species; and
determining that the plant is herbicide-resistant based on the quantification.

54. The method of claim 53, wherein the sample is from Amaranthus palmeri.

55. The method of claim 53, wherein the sample is from a maize, sorghum, wheat, sunflower, rice, soybean, cotton, canola, tobacco, tomato, potato, pepper, barley, alfalfa, sugar cane, or sugar beet plant.

56. The method of claim 53, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide.

57. The method of claim 53, wherein the quantifying expression of the CYP72A219 gene comprises quantifying CYP72A219 mRNA.

58. The method of claim 53, wherein the quantifying expression of the CYP72A219 gene comprises quantifying CYP72A219 polypeptide.

59. The method of claim 53, wherein the sample is a nucleic acid or protein sample.

60. The method of claim 53, wherein the CYP72A219 gene has at least 80%, at least 90%, at least 95%, at least 98%, at least 99%, or 100% sequence identity to SEQ ID NO: 1 or 3.

61. The method of claim 53, wherein the quantifying expression comprises amplifying a nucleic acid using at least two primers.

62. A method for identifying an herbicide-resistant plant, the method comprising:
obtaining a sample from a plant suspected of having herbicide resistance;
detecting one or more motifs in the promoter of a CYP72A219 gene in the sample, wherein the motif comprises the nucleotide sequence of SEQ ID NO: 34, 35, 36, 37, 38, 39, 40, or 41; and
determining that the plant is herbicide-resistant based on the presence of the one or more motifs.

63. The method of claim 62, wherein the CYP72A219 gene has at least 80%, at least 90%, at least 95%, at least 98%, at least 99%, or 100% sequence identity to SEQ ID NO: 1 or 3.

64. The method of claim 62, wherein the detecting comprises amplifying a nucleic acid using at least two primers.

65. A kit for identifying an herbicide-resistant plant, the kit comprising at least two primers, wherein the at least two primers recognize a CYP72A219 gene that is differentially expressed in an herbicide-resistant plant compared to an herbicide-sensitive plant of the same species.

66. The kit of claim 65, wherein the CYP72A219 gene has at least 80%, at least 90%, at least 95%, at least 98%, at least 99%, or 100% sequence identity to SEQ ID NO: 1 or 3.

67. The kit of claim 65, wherein the primers recognize one or more motifs in the promoter of the CYP72A219 gene.

68. The kit of claim 65, further comprising at least one of a positive control and a negative control.

69. The kit of claim 65, further comprising components of a qRT-PCR solution.

70. The kit of claim 65, wherein the plant is Amaranthus palmeri

71. The kit of claim 65, wherein the plant is a maize, sorghum, wheat, sunflower, rice, soybean, cotton, canola, tobacco, tomato, potato, pepper, barley, alfalfa, sugar cane, or sugar beet plant.

72. The kit of claim 65, wherein the herbicide is an HPPD inhibitor herbicide, an ALS inhibitor herbicide, an auxin herbicide, a PPO inhibitor herbicide, an ACCase inhibitor herbicide, a PSII inhibitor herbicide, or a PDS inhibitor herbicide.
